(19) United States
(12) Patent Application Publication
(10) Pub. No.: US 2024/0342179 A1
(43) Pub. Date: Oct. 17, 2024

(54) TREATMENT OF CANCERS OR NEOPLASMS BY INHIBITING KIF15

(71) Applicant: University of Kansas, Lawrence, KS (US)

(72) Inventors: Andrew K. Godwin, Leawood, KS (US); Frank John Schoenen, Lawrence, KS (US); Anuradha Roy, Kansas City, MO (US); Benjamin Gibbs, Kansas City, MO (US); Rebecca Wates, Lees Summit, MO (US)

(21) Appl. No.: 18/635,508

(22) Filed: Apr. 15, 2024

Related U.S. Application Data
(60) Provisional application No. 63/496,270, filed on Apr. 14, 2023.

(51) Int. Cl. A61K 31/519; A61K 31/198; A61K 31/433; A61K 31/4436; A61K 31/506; A61K 31/517; A61K 38/17; A61P 35/00; C12N 15/113

(52) U.S. Cl. CPC A61K 31/519; A61K 31/198; A61K 31/433; A61K 31/4436; A61K 31/506; A61K 31/517; A61K 38/1709; A61P 35/00; C12N 15/113; C12N 2310/14

(57) ABSTRACT

A method of inhibiting KIF15 can include administering a compound to the KIF15 to inhibit biological functionality of KIF15. The compound includes a structure of Formula 1. R¹ and R² are each independently a non-hydrogen substituent, such that the compound inhibits biological functionality of KIF15, or a stereoisomeric form or a mixture of stereoisomeric forms, or pharmaceutically acceptable salts of the compound. The compound is administered to the KIF15 to inhibit KIF15 from interacting with TPX2 so as to inhibit or disrupt a protein-protein interaction therebetween. The administering of the compound can be to a subject having the KIF15, which is administered in a therapeutically effective amount to treat a disease or disorder associated with KIF15 interacting with TPX2, such as treating a cancer or neoplasm.

### CROSS-REFERENCE TO RELATED APPLICATIONS

This patent application claims priority to U.S. Provisional Application No. 63/496,270 filed Apr. 14, 2023, which provisional is incorporated herein by specific reference in its entirety.

### U.S. GOVERNMENT RIGHTS

This invention was made with government support under CA214545 and CA140323 awarded by the National Institutes of Health. The government has certain rights in the invention.

### BACKGROUND

### Field

The present disclosure relates to compounds and methods for inhibiting or disrupting the biological function of KIF15, which can be used as a therapy for cancers or neoplasms.

### Description of Related Art

Cancer continues to be a leading cause of death worldwide. While molecularly targeted drugs and immunotherapy-based agents have made significant inroads over the past decade, chemotherapies remain important agents to treat cancer. Since cancer cells grow and multiply much more quickly than most cells in the body, this class of drugs is used in oncology despite serious side effects. Although a simplistic concept, the genomic and genetic complexity and ever-evolving nature of cancers makes treatment with targeted drugs a challenge and emphasizes the need for therapies that exploit cancer-dependent cellular machinery.

Most women diagnosed with epithelial ovarian cancers (EOCs) are diagnosed at an advanced stage where curative treatment is not an option. The standard-of-care taxane/platinum therapy for women with EOCs leaves much to be desired. The approval of PARP inhibitors for maintenance therapy independent of BRCA-status has expanded the toolbox for oncologists and has improved treatment outcomes. PARP inhibitors increase the time for progression-free survival between lines of active therapy, however, disease progression occurs requiring additional rounds of therapy. With each iterative round of therapy, progression-free survival decreases, cells become more resistant to active therapies, and eventually no approved treatment will lead to a complete response. Unfortunately, the patient will ultimately succumb to the persistent and progressive disease. New approaches to active therapies remain a strong clinical need.

Oncologists have had success treating cancers that target specific genetic aberrations in the tumor (e.g., imatinib targeting BCL-ABL fusion in CML and KIT/PDGFR mutations in GIST) or pathways necessary for tumor progression in specific tumor types (e.g., enzalutamide in prostate cancer). However, the most common and most lethal subtype of EOC, high-grade serous ovarian carcinoma, is driven by loss of function mutations to TP53, resulting in the inactivation of p53, a tumor suppressor protein. p53 mutations continue to evade targeted therapy approaches.

To identify new therapies for both adult and pediatric cancers that are still dependent on chemotherapies (e.g., epithelial ovarian cancer (EOC) and Ewing Sarcoma (EWS), respectively), in silico and drug re-purposing screens were conducted¹⁻⁸ that revealed proteins essential for mitotic spindle formation and cell cycle progression including Kinesin Family Member 11 (KIF11), Kinesin Family Member 15 (KIF15) and its binding partner the targeting protein for Xklp2 (TPX2), and Aurora Kinase A (AURKA). In parallel studies, an RNAi-based screen of the “druggable genome” was performed to identify putative points of molecular vulnerability across a diverse panel of ovarian cancer cell lines⁴. This screen also identified KIF11 as an essential protein in maintaining tumor cell viability. KIF11 (Eg5) is a tetrameric microtubule crosslinker and mitotic motor protein which facilitates mitotic progression through metaphase and anaphase by binding and pushing apart microtubules in the bipolar spindle. KIF11 has also been identified as a viable cancer drug target. Although KIF11 inhibitors have been reported to be well tolerated by patients, the clinical response rates when administered as monotherapies were typically less than 10% in heavily treated patients with advanced disease⁹.

Despite treatments currently available, cancer still has a high mortality rate¹⁰⁻¹².

KIF11 has been a cancer target pursued by the pharmaceutical industry^{9, 13-18}. Under physiological conditions, KIF11 is essential for forming the bipolar spindle during mitosis¹⁹⁻²⁵. It functions in chromosome positioning, centrosome separation and establishing a bipolar spindle during cell mitosis making it a logical target for cancer treatment. Many allosteric inhibitors have been developed targeting the Loop 5 Pocket on the surface of the motor domain of KIF11 and potently inhibit ATP hydrolysis (e.g., IC₅₀ monastrol: 14 μM; STLC: 1 μM; filanesib: 6 nM; ispinesib: 1.7 nM; SB-743921: 100 pM)^{22, 26-33}. Trials have been initiated in several cancer types including heme malignancies (e.g., ALL, CML) and solid tumors (e.g., breast, colorectal, head and neck, lung cancers)³³⁻⁴⁸. Despite the superb characteristics of KIF11 inhibitors (KIF11i), such as ispinesib (i.e., high potency, high specificity, well-tolerated by patients), clinical objective response rates were typically less than 10% in phase II trials.

KIF15 is a 1,388 amino acid plus-end-directed motor capable of switching between processive and diffusive states. KIF15 functions both as a homodimer and homotetramer⁴⁹⁻⁵². The mechanism from which KIF15 compensates for inhibited KIF11 is unknown. However, it is known that the endogenous levels of KIF15 in the presence of rigor KIF11 (i.e., microtubule crosslinking function remains while motor function is inhibited)⁴⁹, or overexpression of KIF15, is necessary for mitosis to occur in a KIF11-motor independent fashion⁵³. KIF15 binds to the targeting protein for Xklp2 (TPX2), a microtubule- and cell cycle-associated protein which facilitates the targeting of KIF15 to spindle microtubule and overexpressed in various malignancies⁵⁴⁻⁵⁶. Once KIF15 has targeted and engaged with TPX2 by the dimerized leucine-zipper motif near the C-terminus of KIF15 (aa1359-1380)^{57, 58}, TPX2 facilitates better microtubule gripping than that of KIF15 alone^{49, 50}.

Other mitotic kinesins have been shown to be viable targets for the treatment of cancer^{18, 59-63}. Although the mechanism is unclear, the KIF11-independent bipolar spindle assembly is dependent on the function of KIF18B and MCAK (mitotic centromere-associated kinesin)⁵⁹⁻⁶¹. Lastly, Aurora kinase A (AURKA), a serine/threonine kinase with crucial functions in mitosis, has aberrant expression in many cancer types^{64, 65}. AURKA-mediated phosphorylation can regulate the functions of AURKA substrates, some of which are mitosis regulators, including KIF15^{59, 66-68} via phosphorylation of serine residue 1,169 in KIF15, which is necessary for KIF15 to engage a spindle load⁶⁶. Other studies have shown differential dependence on mitotic kinesins in the highly stressed bipolar spindle in an aneuploid state^{69, 70}. Together, these studies indicate the superfamily of kinesins as a viable target for the treatment of a wide variety of human malignancies. In particular, the simultaneous inhibition of KIF11 and KIF15 induces mitotic catastrophe leading to cancer cell death.

New treatment approaches for patients with EOC independent of genetic profile and histological subtype is needed. Previously, an RNAi-based screen of the druggable genome was used to identify vulnerabilities that could represent a new therapeutic strategy for cancers. Analysis found that KIF11 was clearly the most potent mediator of ovarian cancer cell viability.

KIF11 is a tetrameric crosslinker and mitotic motor protein which facilitates mitotic progression through metaphase and anaphase by binding and pushing apart microtubules in the bipolar spindle. Numerous KIF11 inhibitors have been developed targeting a unique domain in the protein that binds allosteric modulators. These inhibitors bind the L5 pocket (Tyr125-Glu145; helix a2/loop L5 and helix a3) on the surface of the motor domain and potently inhibit motor ATP hydrolysis (monastrol: 4.9 μM; S-trityl-L-cysteine (STLC): 900 nM), and thus motor function. The allosteric KIF11 inhibitors have K values in the high pM to low nM range (ispinesib: 1.7 nM; SB-743921: 100 pM; filanesib: 6 nM) with no activity against other kinesins or other motor proteins (e.g., KIF1A, KIF15, KIF4, CENP-E, RabK6, MCAK, MKLP1, KHC, KIN2). Unfortunately, clinical trials to date have failed to show efficacy for KIF11 inhibitors as monotherapies despite these drugs being highly specific for KIF11 and well tolerated by patients.

Despite the development of new therapies designed to improve treatment outcomes, epithelial ovarian cancer (EOC) remains the deadliest cancer of the female reproductive tract⁷¹⁻⁸¹. Ewing Sarcoma (EWS), the second-most-common pediatric osseous malignancy, disproportionally afflicts children and young adults⁸². Over the past 30 years, the survival rates for localized EWS have improved considerably from 10% to 70% with multi-agent chemotherapy, yet approximately 30% to 40% of pediatric EWS patients experience recurrence of their disease^{83, 84}. The mitotic spindle formation and cell cycle progression can include the targets KIF11, KIF15, TPX2, and AURKA^{1, 2}, which are upregulated by EWS-FLI1, the most common oncoprotein in EWS⁸⁵. KIF15 cooperates with KIF11 to promote bipolar spindle assembly and formation, which is essential for proper sister chromatid segregation and when genetically silenced, can enhance the efficacy of KIF11 inhibition^{51, 53, 59, 86-88}.

Literature has indicated that combined inhibition of KIF11 and KIF15 could be a promising treatment strategy across many malignant and neoplastic diseases^{89-94, 95-97}. To date, two research-grade KIF15 inhibitors have been described, KIF15-IN-1⁹⁶ and GW108X⁹⁸. Mechanistically, KTF15-IN-1 is a KIF15-specific ATP-competitive inhibitor with the median inhibitory concentration of 200 nM to 1.7 μM. GW108X is an allosteric inhibitor with a median inhibitory concentration of 800 nM with some activity against other kinesins. Both compounds approach inhibiting KIF15 by simply targeting the motor domain.

### SUMMARY

In some embodiments, a method of inhibiting KIF15 is provided. The method of inhibiting KIF15 can include administering a compound to the KIF15 to inhibit biological functionality of KIF15. The compound includes a structure of Formula 1;

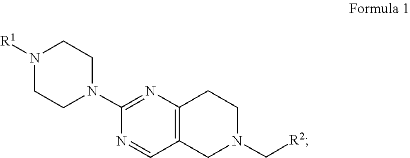

Wherein: R¹ and R² are each independently a non-hydrogen substituent, such that the compound inhibits biological functionality of KIF15, or a stereoisomeric form or a mixture of stereoisomeric forms, or pharmaceutically acceptable salts of the compound.

In some embodiments, the compound is administered to the KIF15 to inhibit KIF15 from interacting with TPX2 so as to inhibit a protein-protein interaction therebetween. In some aspects, the administration of the compound to the KIF15 is in vitro. In some aspects, the administration of the compound to the KIF15 is in vivo. The inhibiting of KIF15, or any other biological protein, can include inhibiting or disrupting biological activity. As such, inhibiting KIF15 can include disrupting biological activity thereof.

In some embodiments, the administration of the compound to the KIF15 is conducted by administering the compound to a subject having the KIF15. In other embodiments, the administration of the compound to the KIF15 is conducted by administering the compound directly to the KIF15 in vitro. The compound can be administered in a therapeutically effective amount for a sufficient length of time to treat a disease or disorder associated with KIF15 interacting with TPX2, such as treating a neoplasm (e.g., malignant neoplasm, such as cancer).

In some embodiments, the method of inhibiting KIF15 can be used in a method of treating cancer. Such a method of cancer treatment can include providing a subject having been diagnosed with cancer and administering the compound to the subject in a therapeutically effective amount for a sufficient length of time to treat the cancer. While the cancer can be any cancer that expresses KIF15 and/or TPX2, the cancer can be selected from breast cancer, colorectal cancer, lung cancer, ovarian cancer, bone cancer, and prostate cancer.

In some embodiments, the method of inhibiting KIF15 can be used in a method of treating a sarcoma. Such a method of sarcoma treatment can include providing a subject having been diagnosed with a sarcoma and administering the compound to the subject in a therapeutically effective amount for a sufficient length of time to treat the sarcoma. While the sarcoma can be any sarcoma that expresses KIF15 and/or TPX2, the sarcoma can be Ewing sarcoma (EWS), osteosarcoma, chondrosaroma, fibrosarcoma, synovial sarcoma, rhabdomysarcoma, angiosarcoma, or others.

In some embodiments, the method of inhibiting KIF15 can be used in a method of treating a neoplasm. Such a method of neoplasm treatment can include providing a subject having been diagnosed with a neoplasm and administering the compound to the subject in a therapeutically effective amount for a sufficient length of time to treat the neoplasm. While the neoplasm can be any type of neoplasm, whether benign or cancer, the neoplasm expresses KIF15.

In some embodiments, any of the methods recited herein can also include inhibiting KIF11. As such, the methods can include the combination of inhibiting KIF11 and KIF15. The method can inhibit KIF15 with the compound described herein. The KIF11 can be inhibited by administering a second compound that inhibits biological functionality of KIF11. The second compound can be administered at the same time, in the same formulation, at different times, and/or in different formulations with respect to the compounds of the invention that are KIF15 inhibitors. For example, the second compound includes an siRNA for KIF11, monastrol, Eg5 inhibitors, S-trityl-L-cysteine, ispinesib, filanesib, or a tail domain of KIF11 peptide, or other KIF11 inhibitor.

In some embodiments, a method of treating a cancer or neoplasm can include: inhibiting biological functionality of KIF11 in a subject having cancer; and inhibiting biological functionality of KIF15 in the subject with a compound comprising a structure of Formula 1, as shown herein.

In some embodiments, a kit for treating cancer or neoplasm can include a compound of Formula 1 and a KIF11 inhibitor, such as one described herein or otherwise developed.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1A includes images that show slides that were stained for KIF15, KIF11, TPX2 and/or hematoxylin & eosin the scanned and analyzed using an Ariol system and HALO software.

FIG. 1B includes graphs that show H-scores that were calculated for staining intensity of each protein in each tissue, stratified based on histotypes.

FIG. 1C shows immunoblotting gels from cell lysates (20 μg total protein) that were collected from nine ovarian cancer cell lines, two immortalized ovarian epithelial cell lines and two fallopian tube epithelial cell lines in RIPA buffer, then electrophoresed, transferred to PVDF, and immunoblotted for KIF11 (polyclonal Rabbit, Cell Signaling), KIF15 (Abcam), TPX2 (Cell Signaling) or GAPDH (Abcam).

FIG. 1D includes the immunoblotting gel for tumor lysates (20 μg of total protein) and lysates from cancer-free ovaries, which were evaluated by immunoblotting for KIF11 (Cell Signaling) and KIF15 (Abcam).

FIG. 2A shows a bar graph with data for the GL2 (negative control), PLK (positive control), or KIF15 were knocked down by siRNA in 4 ovarian cancer cell lines, A2780, CP70, OVCAR5, SKOV3.

FIGS. 2B-2E include graphs of data showing the viability of the cell line versus ispinesib.

FIGS. 2F-2K show data that the knocking down of KIF15 re-sensitizes ispinesib-resistant ovarian cancer cell lines to KIF11 inhibitors (ispinesib, SB-743921, ARRY520).

FIG. 3A shows Compound A1, Compound A2, and Compound B1.

FIG. 3B includes a graph that shows the inhibition percentage by Compound A1 versus concentration.

FIG. 3C includes a graph that shows the KIF15 inhibition percentage by Compound A2 versus concentration.

FIG. 3D includes a graph that shows the KIF15 inhibition percentage by Compound B1 versus concentration.

FIG. 4A shows the NMR spectra of Compound A1.

FIG. 4B shows the NMR spectra of Compound B1.

FIG. 4C includes a graph with melt curves of KIF15-HiBiT thermally challenged for 5 minutes in the presences of 100 μM of Compound A1, A2, B1 or TPX2 mimetic peptide.

FIG. 5A includes a diagraph showing the Bliss Score for Compound A1 regarding ispinesib.

FIG. 5B includes a diagraph showing the Bliss Score for Compound A2 regarding ispinesib.

FIG. 5C includes a diagraph showing the Bliss Score for Compound B1 regarding ispinesib.

FIG. 6A-6P include graphs that show the percent inhibition of KIF15 versus concentration of inhibitor, as identified by the hit number shown in Table 1.

FIG. 7 shows the data for a screen of cocktail #4, which shows hits.

FIG. 8 shows the data for a screen of cocktail #7, which shows hits.

FIG. 9 shows the structure-activity relationship using WLOG.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes compounds and/or materials for use as inhibitors of KIF15. These inhibitors may, without being bound thereto, inhibit an interaction between KIF15 and TPX2. Thus, the inhibitors may be characterized by inhibiting the KIF15-TPX2 protein-protein interaction. In some aspects, the compounds described herein provide more efficacious therapeutic approaches for patients with cancer, focusing on the KIF11/KIF15/TPX2 biological pathways. The compounds include chemotypes that disrupt KIF15-TPX2 binding for inhibition of KIF15's primary functions, such as antiparallel microtubule sliding. This inhibition of KIF15 can abrogate its compensatory function in the presence of KIF11 and KIF11 inhibitors, and thereby improve the inhibitory effect of inhibiting KIF11 when KIF15 is inhibited. In combination, inhibitors targeting both KIF11 and KIF15 can stall mitosis and further sensitize cancers cells to current cytotoxic treatments.

Information about KIF15 is provided by Wikipedia, which provides for identification of the protein (wikipedia.org/wiki/KIF15), which is incorporated herein by specific reference in its entirety.

Information about KIF11 is provided by Wikipedia, which provides for identification of the protein (wikipedia.org/wiki/Kinesin-like_protein_KIF11), which is incorporated herein by specific reference in its entirety.

Information about TPX2 is provided by Wikipedia, which provides for identification of the protein (wikipedia.org/wiki/TPX2), which is incorporated herein by specific reference in its entirety.

An AlphaScreen-based high-throughput screening assay can be used to identify inhibitors of the KIF15-TPX2 protein-protein interaction. These inhibitors have been followed up with cell independent and cell dependent assays to validate inhibitor activity. From this, we have identified two chemotypes with putative inhibitors of the KIF15-TPX2 protein-protein interaction. Analogs of one of the compounds have been synthesized to allow compounds to have better drug-like properties.

In some embodiments, the current invention deals with the molecules referred to as compound A1, compound A2 and compound B1 for the treatment of neoplasms, whether benign or cancer, by interference of the TPX2-KIF15 protein-protein interaction (PPI). These small molecules were each identified as a KIF15/TPX2 PPI inhibitor as part of a high throughput screen. In some aspects, these molecules are inhibitors of KIF15-TPX2 PPI for the treatment of neoplasms, including malignant neoplasms or cancer. Thus, the compounds and methods can be used for a cancer therapy, such as by inhibiting development and progression of cancer. While treatment of cancer is described as an example herein, such examples show treatment of a neoplasm with the molecules described herein.

In some embodiments, a compound is provided to inhibit biological functionality of KIF15. The compound includes a structure of Formula 1;

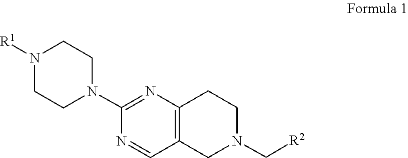

Wherein: R¹ and R² are each independently a non-hydrogen substituent, such that the compound inhibits biological functionality of KIF15, or a stereoisomeric form or a mixture of stereoisomeric forms, prodrug, or pharmaceutically acceptable salts of the compound.

In some embodiments, the R¹ and R² each independently include halogens, hydroxyls, alkoxys, straight aliphatics, branched aliphatics, cyclic aliphatics, substituted aliphatics, unsubstituted aliphatics, saturated aliphatics, unsaturated aliphatics, aromatics, polyaromatics, substituted aromatics, hetero-aromatics, hetero-polyaromatics, substituted hetero-polyaromatics, amines, primary amines, secondary amines, tertiary amines, aliphatic amines, carbonyls, carboxyls, amides, esters, phosphates, alkyl phosphates, phosphonate, alkyl phosphonate, carbamates, alkyl carbamates, amino alkyl carbamates, amino acid carbamates, amino acids, peptides, polypeptides, any aryl or cyclo with or without hetero atoms, each being substituted or unsubstituted, or combinations thereof.

In some embodiments, R¹ and R² each independently include an alkyl, alkenyl, alkynyl, cycloalkyl, cycloalkenyl, cycloalkynyl, aryl, polyaryl, hetroaryl, polyhetroaryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof.

In some embodiments, the R¹ and R² each independently can include any one or more of the substituents selected from the group of, C₁-C₂₄ alkyl, C₂-C₂₄ alkenyl, C₂-C₂₄ alkynyl, C₃-C₂₄ cycloalkyl, C₄-C₂₄ cycloalkenyl, C₅-C₂₄ cycloalkynyl, C₅-C₂₀ aryl, C₆-C₂₄ alkaryl, C₆-C₂₄ aralkyl, halo, hydroxyl, sulfhydryl, C₁-C₂₄ alkoxy, C₂-C₂₄ alkenyloxy, C₂-C₂₄ alkynyloxy, C₅-C₂₀ aryloxy, acyl (including C₂-C₂₄ alkylcarbonyl (—CO-alkyl) and C₆-C₂₀ arylcarbonyl (—CO-aryl)), acyloxy (—O-acyl), C₂-C₂₄ alkoxycarbonyl (—(CO)—O-alkyl), C₆-C₂₀ aryloxycarbonyl (—(CO)—O-aryl), halocarbonyl (—CO)—X where X is halo), C₂-C₂₄ alkylcarbonato (—O—(CO)—O-alkyl), C₆-C₂₀ arylcarbonato (—O—(CO)—O-aryl), carboxy (—COOH), carboxylato (—COO⁻), carbamoyl (—(CO)—NH₂), mono-(C₁-C₂₄ alkyl)-substituted carbamoyl (—(CO)—NH(C₁-C₂₄ alkyl)), di-(C₁-C₂₄ alkyl)-substituted carbamoyl (—(CO)—N(C₁-C₂₄ alkyl)₂), mono-substituted arylcarbamoyl ((CO)—NH-aryl), di-substituted arylcarbamoyl (—(CO)—NH-aryl)₂, thiocarbamoyl (—(CS)—NH₂), mono-(C₁-C₂₄ alkyl)-substituted thiocarbamoyl (—(CS)—NH(C₁-C₂₄ alkyl)), di-(C₁-C₂₄ alkyl)-substituted thiocarbamoyl (—(CS)—N(C₁-C₂₄ alkyl)₂), mono-substituted arylthiocarbamoyl (—(CS)—NH-aryl), di-substituted arylthiocarbamoyl (—(CS)—NH-aryl)₂, carbamido (—NH—(CO)—NH₂), ), mono-(C₁-C₂₄ alkyl)-substituted carbamido (—NH—(CO)—NH(C₁-C₂₄ alkyl)), di-(C₁-C₂₄ alkyl)-substituted carbamido (—NH—(CO)—N(C₁-C₂₄ alkyl)₂), mono-substituted aryl carbamido (—NH—(CO)—NH-aryl), di-substituted aryl carbamido (—NH—(CO)—N-(aryl)₂), carbamate (—O—(CO)—NH—), alkyl carbamate (—O—(CO)—NH-alkyl), cyano (—C≡N), isocyano (—N⁺≡C⁻), cyanato (—O—C≡N), isocyanato (—O—N⁺≡C⁻), thiocyanato (—S—C≡N), isothiocyanato (—S—N⁺≡C⁻), azido (—N═N⁺═N⁻), formyl (—(CO)—H), thioformyl (—(CS)—H), amino (—NH₂), mono- and di-(C₁-C₂₄ alkyl)-substituted amino, mono- and di-(C₆-C₂₀ aryl)-substituted amino, C₂-C₂₄ alkylamido (—NH—(CO)-alkyl), C₅-C₂₀ arylamido (—NH—(CO)-aryl), imino (—CR═NH where R is hydrogen, C₁-C₂₄ alkyl, C₅-C₂₀ aryl, C₆-C₂₄ alkaryl, C₆-C₂₄ aralkyl, etc.), alkylimino (—CR═N(alkyl), where R═hydrogen, C₁-C₂₄ alkyl, aryl, alkaryl, aralkyl, etc.), arylimino (—CR═N(aryl), where R═hydrogen, alkyl, aryl, alkaryl, etc.), nitro (—NO₂), nitroso (—NO), sulfonic acid (—SO₂—OH), sulfonato (—SO₂—O⁻) C₁-C₂₄ alkylsulfanyl (—S-alkyl; also termed “alkylthio”), C₅-C₂₀ arylsulfanyl (—S-aryl; also termed “arylthio”), C₁-C₂₄ alkylsulfinyl (—(SO)-alkyl), C₅-C₂₀ arylsulfinyl (—(SO)-aryl), C₁-C₂₄ alkylsulfonyl (—SO₂-alkyl), C₅-C₂₀ arylsulfonyl (—SO₂-aryl), phosphono (—P(O)(OH)₂), phosphonato (—P(O)(O⁻)₂), phosphinato (—P(O)(O—)), phospho (—PO₂), phosphino (—PH₂), phosphate, sulphate, any with or without hetero atoms (e.g., N, O, P, S, or other) where the hetero atoms can be substituted (e.g., hetero atom substituted for carbon in chain or ring) for the carbons or in addition thereto (e.g., hetero atom added to carbon chain or ring) swapped, any including straight chains, any including branches, and any inducing rings, derivatives thereof, and combinations thereof.

In some embodiments, the Formula 1 is defined by the following: R¹ includes a C1-C24 alkyl; R² includes a phenyl, thiophenyl, thiazolyl, imidazolyl, furanyl, pyrrolyl, pyridinyl, pyridazinyl, quinolinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, or morpholinyl, and quinuclidinyl, any substituted or unsubstituted with at least one R³; each R³ independently includes an alkyl, alkenyl, alkynyl, cycloalkyl, cycloalkenyl, cycloalkynyl, aryl, polyaryl, hetroaryl, polyhetroaryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof.

In some embodiments, Formula 1 is defined as follows: R¹ includes a C1-C6 alkyl; R² includes a phenyl, thiophenyl, furanyl, pyrrolyl, pyridinyl, or pyridazinyl, which is substituted with at least one R³; and each R³ is independently fluorine, chlorine, bromine, methyl, ethyl, propyl, isopropyl, butyl, hexyl, methoxy, ethoxy, propoxy, isopropoxy, butoxy, hexoxy, methyl ether, ethyl ether, propyl ether, isopropyl ether, butyl ether, or hexyl ether.

In some embodiments, Formula 1 is defined as follows: R¹ is methyl, ethyl, or propyl; R² includes phenyl or thiophenyl, which is substituted with at least one R³; each R³ is independently fluorine, chlorine, bromine, methyl, ethyl, methoxy, ethoxy, propoxy, or isopropoxy.

In some embodiments, R¹ is methyl, ethyl, or propyl; and the R² includes phenyl and there are 1, 2, or 3 R³ groups. Each R³ is independently fluorine, chlorine, bromine, methyl, ethyl, methoxy, or ethoxy.

In some embodiments, R¹ is methyl, ethyl, or propyl; and the R² includes thiophenyl, and there is one R³ group. The R³ is fluorine, chlorine, or bromine.

In some embodiments, the compounds can include the following structures:

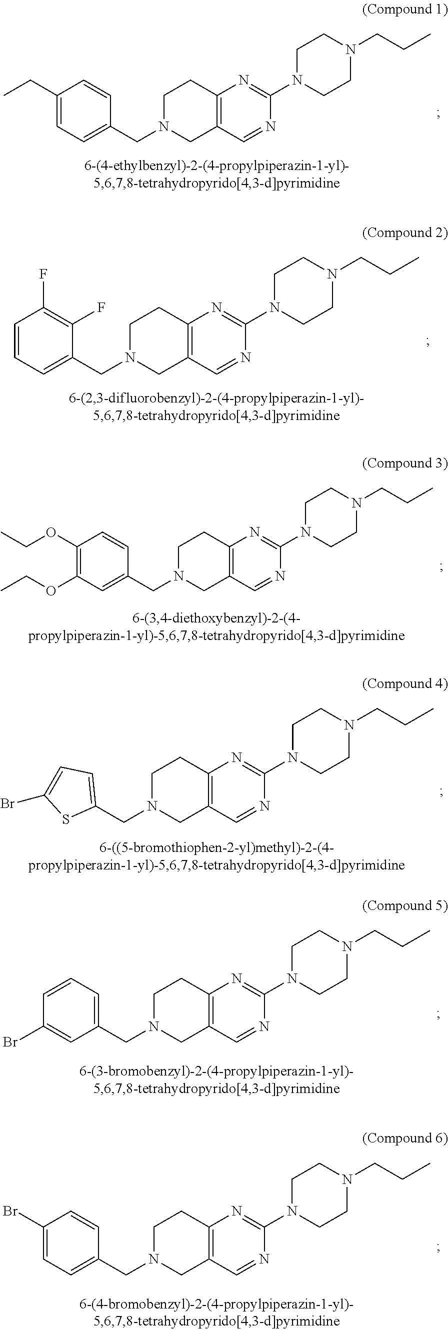

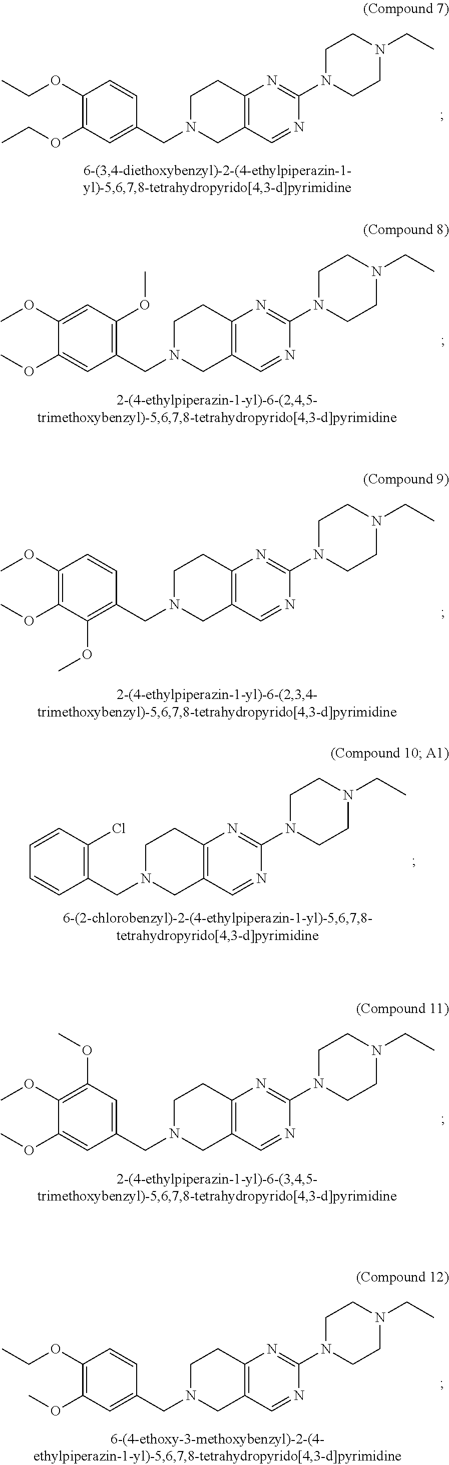

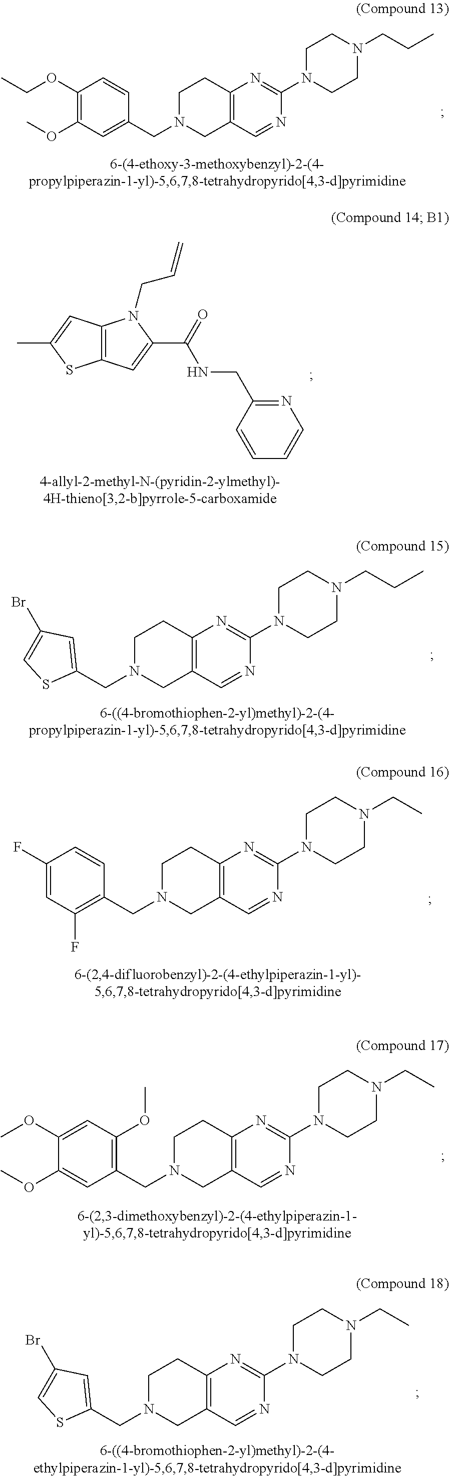

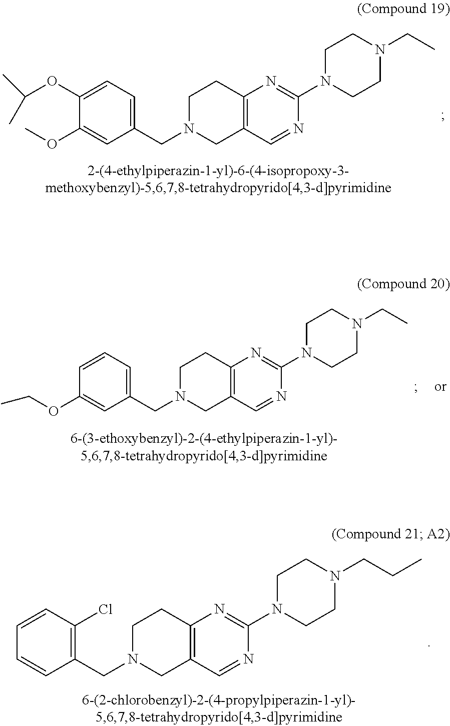

Applicant submits that the substituents on the rings, such as the halo, methyl, ethyl, or propyl, alkoxy, may be replaced by any R group substituent, which can vary. These R groups can be as defined herein. Any of the compounds can be in a stereoisomeric form or a mixture of stereoisomeric forms, prodrugs, or pharmaceutically acceptable salts of the compound.

As used herein, the term “pharmaceutically acceptable salt” refers to those salts which are suitable to use within mammals and do not tend to be toxic. Pharmaceutically acceptable salts are formed using inorganic and organic acids and bases. Examples of pharmaceutically acceptable salts are salts of an amino group formed with inorganic acids such as hydrochloric acid, hydrobromic acid, phosphoric acid, sulfuric acid and perchloric acid, or with organic acids such as tartaric acid, acetic acid, oxalic acid, maleic acid, citric acid, succinic acid or malonic acid, terephthalic acid. Other pharmaceutically acceptable salts include adipate, ascorbate, aspartate, benzoate, bisulfate, borate, butyrate, valerate, camphorate, camphorsulfonate, cyclopentanepropionate, formate, citrate, oxalate, pivalate, succinate, tartrate, fumarate, gluconate, hemisulfate, heptanoate, hexanoate, hydroiodide, 2-hydroxy-ethanesulfonate, lactate, laurate, lauryl sulfate, maleate, malonate, methanesulfonate, 2-naphthalenesulfonate, nicotinate, nitrate, oleate, palmitate, stearate, undecanoate, alginate, 3-phenylpropionate, phosphate, sulfate, thiocyanate, p-toluenesulfonate, benzenesulfonate, persulfate, ethanesulfonate, dodecylsulfate, and the like and mixture salts.

Salts derived from appropriate bases include alkali metal, alkaline earth metal, ammonium and N(Calkyl) salts. Representative alkali or alkaline earth metal salts include Sodium, lithium, potassium, calcium, magnesium, and the like. Further pharmaceutically acceptable salts include, when appropriate, nontoxic ammonium, quaternary ammonium, and amine cations formed using counterions, such as halide, hydroxide, carboxylate, sulfate, phosphate, nitrate, alkyl sulfonate and aryl sulfonate.

In yet another aspect of the present invention, a pharmaceutical composition is provides comprising one or more compounds as indicated above or a salt thereof, and a pharmaceutically acceptable carrier or diluent.

In some embodiments, parenteral administration of the compounds is provided. The term “parenteral” as used herein includes subcutaneous, intravenous, intramuscular, etc. In some embodiments, the compositions are to be administered orally, transdermally, sublingual, buccal, topical, inhalation, nasal, opthalmic, otic, rectal, vaginal, or other routes of administration.

Pharmaceutically acceptable compositions of this invention are orally administered in any orally acceptable dosage form. Exemplary oral dosage forms are capsules, tablets, aqueous suspensions, or solutions. In the case of tablets for oral use, carriers commonly used include lactose and corn starch. Lubricating agents, such as magnesium stearate, are also typically added. For oral administration in a capsule form, useful diluents include lactose and dried cornstarch.

Pharmaceutically acceptable compositions of this invention comprising the compounds are also administered topically, especially when the target of treatment includes areas or organs readily accessible by topical application, including diseases of the eye, the skin, or the lower intestinal tract. Suitable topical formulations are readily prepared for each of these areas or organs.

For topical applications, provided pharmaceutically acceptable compositions are formulated in a suitable ointment wherein the compound of the invention, optionally with other active components, is suspended or dissolved in one or more carriers. Exemplary carriers for topical administration of compounds of this are mineral oil, propylene glycol, polyoxyethylene and water. Suitable topical carriers include, but are not limited to, mineral oil, sorbitan monostearate, polysorbates, cetyl alcohol, benzyl alcohol and water.

Pharmaceutically acceptable compositions of this invention are optionally administered by nasal aerosol or inhalation. Such compositions are prepared according to techniques well-known in the art of pharmaceutical formulation and are prepared as solutions in saline, employing certain conservants, including benzyl alcohol or other suitable preservatives, and/or other conventional solubilizing or dispersing agents.

The amount of the compounds of the present invention of the present invention that are optionally combined with the carrier of vehicle materials to produce a composition in a single dosage form for treating a subject will vary depending upon the patient treated, the mode of administration. Preferably, provided compositions should be formulated so that a dosage of between 0.01-100 mg/kg body weight/day of the compound can be administered to a patient receiving these compositions.

The compounds of the present invention can also be in micro-encapsulated form with one or more excipients as noted above. The solid dosage forms of tablets, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose, or starch. Such dosage forms also comprise, as is normal practice, additional substances other than inert diluents, e.g., tableting lubricants and other tableting aids such a magnesium stearate and microcrystalline cellulose. In the case of capsules, tablets and pills, the dosage forms optionally also comprise buffering agents. They optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Additionally, muti-layered dosage forms can be used, which can provide for modified and controlled release of the drug from the oral formulation. The dosage form may be configured with pH sensitive moieties or other stimuli sensitive moieties to selectively and controllably release the formulation in the desired location, whether stomach, small intestine or large intestine.

Dosage forms for topical or transdermal administration of a compound of this invention include ointments, pastes, creams, lotions, gels, powders, solutions, sprays, inhalants, or patches. The active component is admixed under sterile conditions with a pharmaceutically acceptable carrier and any needed preservatives or buffers as required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### EXAMPLES

### KIF11, KIF15 and TPX2 Expression in Ovarian Tumors and Cell Lines

The ovarian cancer (OC) cell lines were grown in RPMI supplemented with 10% FBS (Atlanta Biologicals), 2 mM L-glutamine (Invitrogen), 7.5 μg/mL insulin, 100 IU/mL penicillin (Invitrogen), and 100 μg/mL streptomycin (Invitrogen). Two ovarian cancer cell lines, OVCAR5 and SKOV3, were cultured in increasing concentrations of ispinesib (5 nM-1 μM) to develop KIF11 inhibitor resistance. The most resistant lines (selected in the presence of 1 μM ispinesib) were named OVCAR5-ISP^{R} and SKOV3-ISP^{R}, respectively. Immortalized surface epithelial cell lines were maintained in MCDB105/Medium 199 (Gibco, Thermo Fisher) 1:1 supplemented with 5% FBS (Atlanta Biologicals), 0.25 units/mL insulin, 100 IU/mL penicillin (Invitrogen), and 100 μg/mL streptomycin (Invitrogen). Immortalized fallopian tube epithelial cell lines FT190 and FT194 were maintained in DMEM/Ham's F-12 (Gibco, Thermo Fisher)) 1:1 supplemented with 2% Ultroser G Serum Substitute (Pall Life Sciences), 100 IU/mL penicillin (Invitrogen), and 100 μg/mL streptomycin (Invitrogen). All cell lines were maintained at 37° C., 5% CO₂ in a humidified incubator. Authentication: cell lines were authenticated by IDexx Bio, Columbia, MO and mycop/asma testing was performed using the LookOut Myco PCR test (Sigma Aldrich, St. Louis, MO).

Tissue microarrays (TMAs) were constructed from archival formalin-fixed, paraffin embedded samples of ovarian carcinoma (49 patients) along with matched metastases and recurrences (15 patients; 2 of these 15 patients had 2 recurrences each), matched metastases alone (27 patients) and matched recurrences alone (7 patients). Based on review of the original pathology reports, the ovarian carcinomas were typed as serous (31 samples), mixed (14 samples; 12 of which included a serous component), carcinosarcoma (1 sample), clear cell (1 sample), papillary carcinoma, not otherwise specified (NOS) (1 sample) and adenocarcinoma, and NOS (1 sample). For every sample, hematoxylin- and eosin-stained slides were reviewed by a board-certified pathologist who selected tumor rich areas. Using the semi-automated TMArrayer (Pathology Devices, Inc., Westminster, MD) TMA paraffin blocks were assembled with triplicate 1.0 mm cores using the marked slide as a guide. Antibodies for KIF11 (23333-1-AP) from Proteintech (Rosemont, IL), KIF15 (55407-1-AP) from Proteintech (Rosemont, IL), and TPX2 (ab32795) from Abeam (Cambridge, MA) were used for immunohistochemical staining according to the following procedure. Four-micron paraffin sections are mounted on Fisherbrand Superfrost slides and baked for 60 minutes at 60° C. then deparaffinized. Epitope retrieval was performed in a Biocare Decloaking Chamber (pressure cooker), under pressure for 5 minutes, using pH 6.0 citrate buffer followed by a 10-minute cool-down period. Endogenous peroxidase is blocked with 3% H₂O₂ for 10 minutes followed by incubation with primary antibody for 45 minutes (KIF11, 1:200, 45 minutes; KIF15, 1:1000, 30 minutes; TPX2, 1:200, 30 minutes). Next, slides were incubated with Mach 2 HRP Polymer BioCare (Concord, CA.) for 30 minutes and DAB+ chromogen (Dako, Carpinteria, CA.) for 5 minutes. Immunohistochemical staining was performed using the IntelliPATH FLX Automated Stainer at room temperature. A light hematoxylin counterstain was performed, following which the slides were dehydrated, cleared, and mounted using permanent mounting media. Immunostaining was quantified by HALO 2.0 next-generation digital pathology (Indica Labs). Epithelial tumor components were annotated and then staining intensity (0, 1+, 2+, 3+) was measured across the epithelial tumors for each KIF11, KIF15, and TPX2. The KIF11 and KIF15 stainings were measured in the cytoplasm and nucleus and TPX2 staining was quantified only in the nuclear compartment.

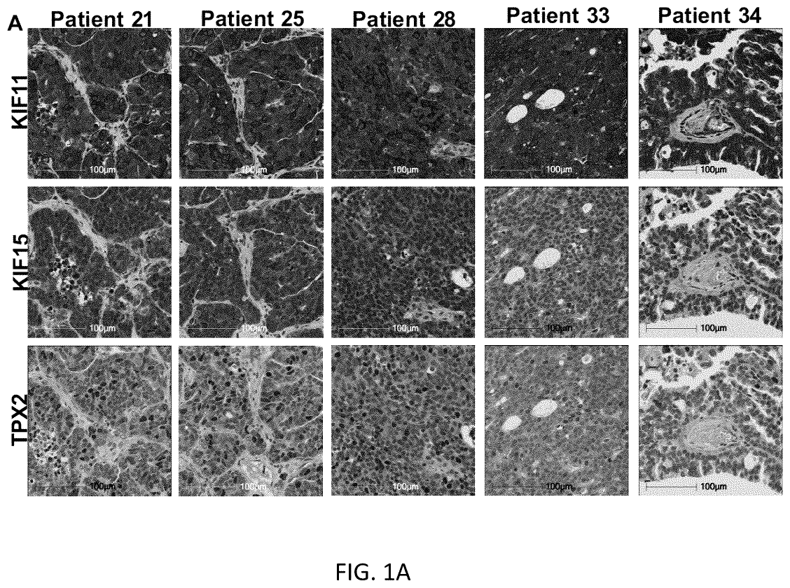

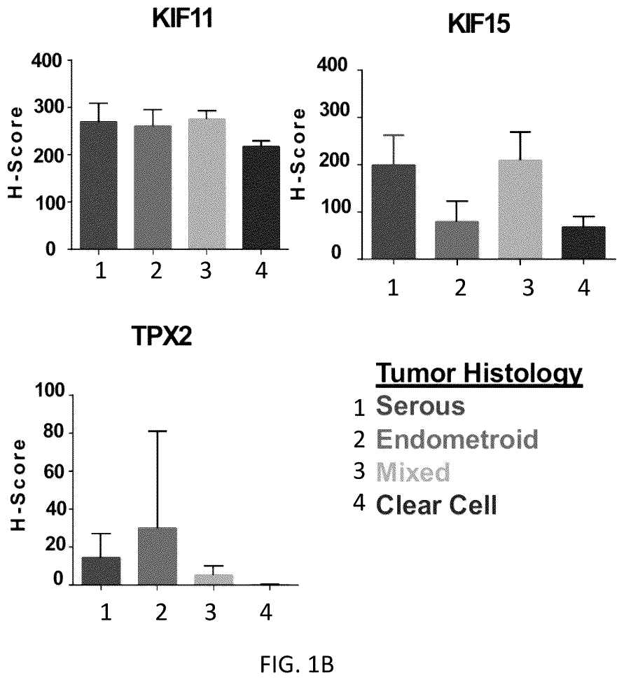

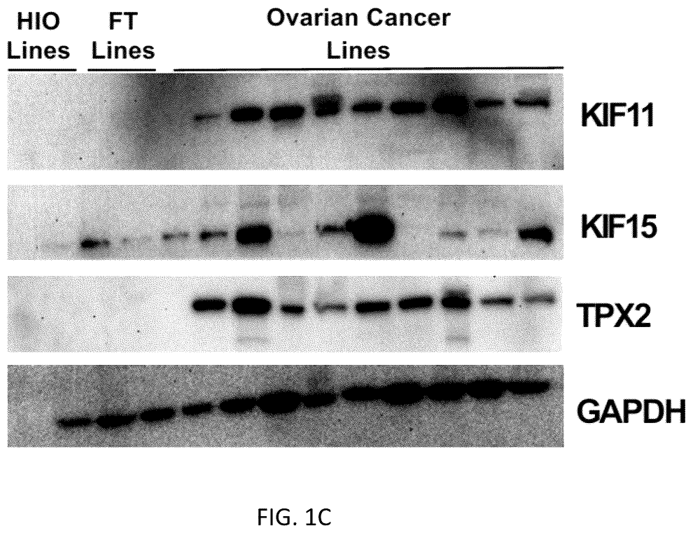

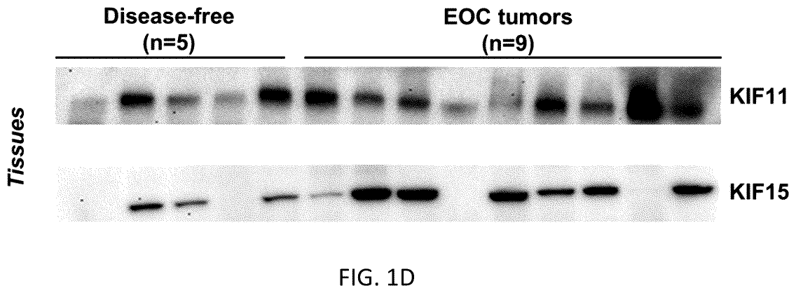

Primary, metastatic, and recurrent tumors from forty-nine patients were compiled to create a tissue microarray block (only primary tumors were included in this study). Tissues (four-micron sections) were mounted onto slides and prepared for staining. Slides were stained for KIF15, KIF11, TPX2 and/or hematoxylin & eosin the scanned and analyzed using an Ariol system and HALO software, as shown in FIG. 1A. H-scores were calculated for staining intensity of each protein in each tissue, stratified based on histotypes, and are reported in bar graphs as shown in FIG. 1B.

Cells lines were cultured to 70% confluence, then lysed in RIPA buffer (Thermo Fisher, Waltham, MA) supplemented with protease (Complete Mini, Roche, Basel, Switzerland) and phosphatase inhibitor cocktails (Thermo Fisher, Waltham, MA). Cryopreserved tissues were disrupted in RIPA buffer using a Bullet Blender tissue homogenizer and the Green Eppendorf Lysis Kit (Next Advance, Averill Park, NY). Protein concentrations were measured using a standard Bradford assay, and 20 μg of total protein was electrophoresed on a 4-20% polyacrylamide gel (BioRad, Hercules, CA), then transferred to a PVD F membrane and immunoblotted as indicated. Rabbit anti-KIF15 and rabbit anti-TPX2 were purchased from Proteintech (Rosemont, IL). Rabbit anti-KIF11(Eg5), HRP-conjugated anti-b-actin, and anti-rabbit HRP-conjugated secondary antibody were purchased from Cell Signaling Technology (Danvers, MA). HRP-conjugated rabbit anti-GAPDH was purchased from Abcam (Cambridge, UK). Where indicated, cells were treated with targeting siRNAs for 48 hours followed by lysis, electrophoresis, and immunoblotting.

FIG. 1C shows immunoblotting gels from cell lysates (20 μg total protein) that were collected from nine ovarian cancer cell lines, two immortalized ovarian epithelial cell lines and two fallopian tube epithelial cell lines in RIPA buffer, then electrophoresed, transferred to PVDF and immunoblotted for KIF11 (polyclonal Rabbit, Cell Signaling), KIF15 (Abcam), TPX2 (Cell Signaling) or GAPDH (Abcam). Membranes were exposed and images captured using a Fluor ChemE Imaging System (Protein Simple, San Jose, CA). FIG. 1D includes the immunoblotting gel for tumor lysates (20 μg of total protein) and lysates from cancer-free ovaries, which were evaluated by immunoblotting for KIF11 (Cell Signaling) and KIF15 (Abcam).

The protocol measured KIF11, KIF15 and TPX2 protein expression in 48 clinical ovarian carcinoma tissue specimens of varying histotypes (serous, endometrioid, clear cell, mixed) and 1 primary peritoneal carcinoma and found that KIF11 is ubiquitously expressed (nuclear and cytoplasmic) in these tumors, regardless of clinical subtype. The protocol also observed that KIF15 is ubiquitously expressed (nuclear and cytoplasmic), although higher protein levels are detected in the serous and mixed histotypes. TPX2 exhibits intense nuclear staining at varying levels across the histotypes. H-scores were calculated and are reported in bar graphs in FIG. 1B. The protocol also evaluated a panel of cell lines derived from fallopian tube, ovarian epithelium or ovarian tumors from multiple ovarian cancer subtypes. Like our observations in stained sections, ubiquitous expression was observed of all three proteins in tumor-derived cell lines.

### KIF15 Compensates for KIF11 in the Presence of KIF11 Inhibitors in Ovarian Cancer Cell Lines

Cells (3,000 cells/well) were plated in supplemented media in 96-well black plates (clear bottom) and allowed to adhere overnight at 37° C., 5% CO₂. Medium was removed and replaced with fresh supplemented media containing ispinesib (Selleck Chemicals), SB-743,921 (Selleck Chemicals), or filanesib (MedChemExpress) in concentrations ranging from 0.49 nM-1 μM as appropriate. After 72 hours, cell viability was measured according to manufacturer's recommendation using the luminescent CellTiter Glo reagent diluted 1:3 in Glo Lysis buffer (100 μl/well; Promega). Where indicated, cells were first incubated with 50 nmol siRNA pool targeting KIF15, TPX2 or PLK1 (negative control) or a single siRNA targeting GL2 (negative control). Reverse transcription was achieved by diluting siRNAs (final concentration 50 nM) with Lipofectamine RNAiMax reagent (Invitrogen) in reduced-serum media (OptiMEM, Invitrogen) to form lipid complexes. Following a 30-minute incubation at room temperature the lipid complexes were added to cells overnight (37° C., 5% CO₂). Fresh drug-containing media was added for a 72-hour incubation as described above.

The protocol determined KIF11i activity in a panel of ovarian cancer cell lines by treating nine cancer cell lines and 4 non-tumor cell line models to increasing concentrations (0.49-250 nM) of a representative KIF11i, ispinesib. The data shows greater than 50% loss of viability in all 12 cancer cell lines with calculated IC50 values ranging from 1.07 nM-15.83 nM. The data shows cytotoxicity in the immortalized, non-tumorigenic cells lines representing ovarian surface epithelium (HIO-80, HIO-118) and fallopian tube epithelium (FT190, FT194). The calculated IC50 values for these control cell lines ranged from 7.04-23.45 nM.

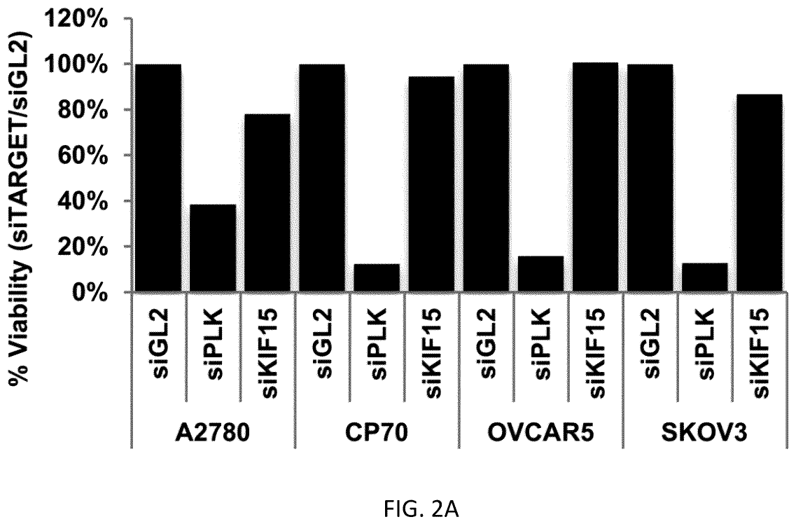

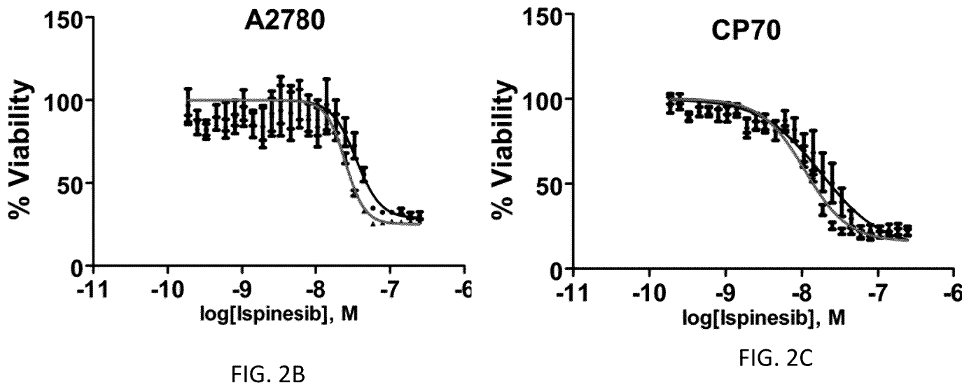

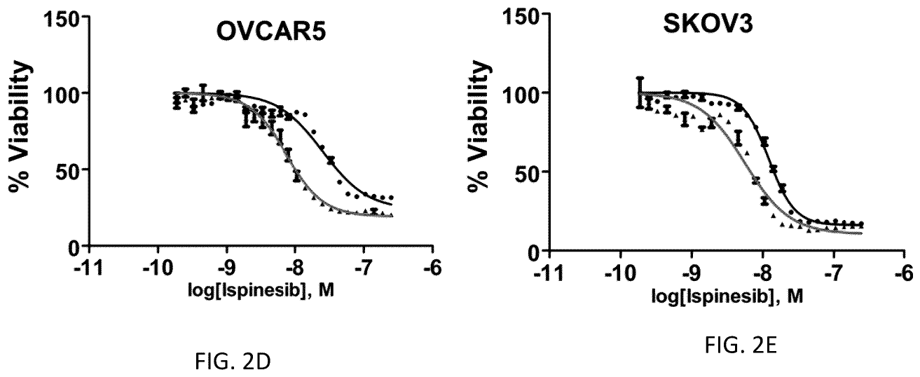

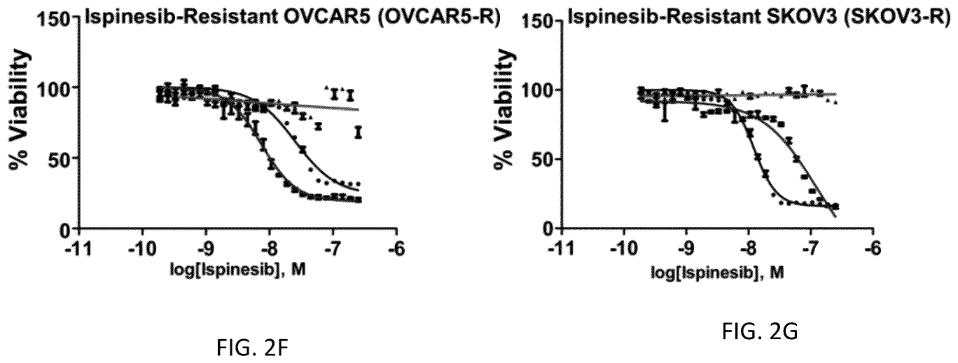

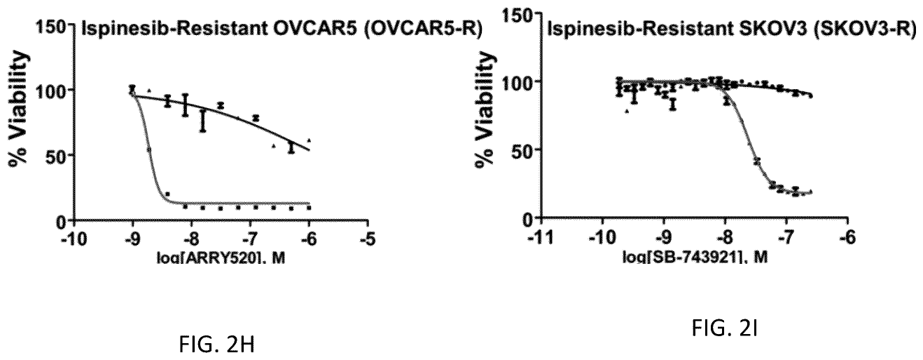

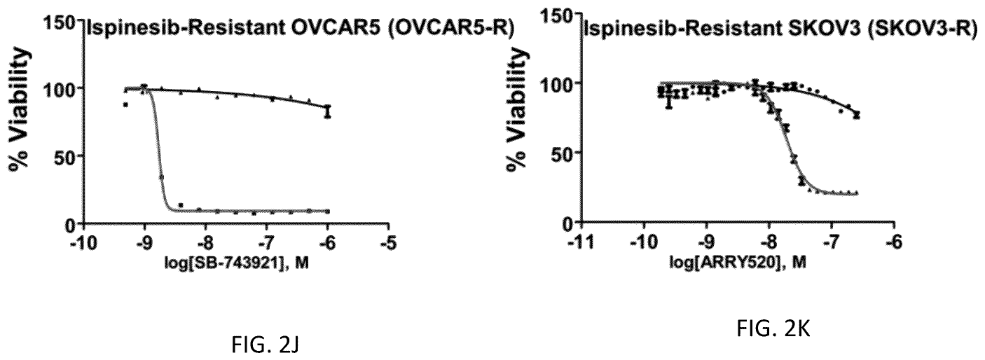

FIG. 2A shows a bar graph with data for the GL2 (negative control), PLK (positive control), or KIF15 were knocked down by siRNA in 4 ovarian cancer cell lines, A2780, CP70, OVCAR5, SKOV3. As expected, the silencing of KIF15 alone has little effect on the viability of cells. FIGS. 2B-2E include graphs of data showing the viability of the cell line versus ispinesib. The data shows that knocking down KIF15 increases the sensitivity of ovarian cancer cell lines to ispinesib treatment. Accordingly, the inhibition of KIF15 can be performed to increase effectiveness of ispinesib in cancer treatment. FIGS. 2F-2K show data that the knocking down of KIF15 re-sensitizes ispinesib-resistant ovarian cancer cell lines to KIF11 inhibitors (ispinesib, SB-743921, ARRY520). This further supports the combination treatment to inhibit KIF11 and KIF15.

First, the protocol evaluated whether loss of KIF15 or TPX2 alone can lead to tumor cell death. The protocol treated 4 cell lines (A2780, CP70, OVCAR-5, SKOV3), representing variable relative expression of KIF11/KIF15/TPX2, with pooled siRNAs targeting either KIF15, TPX2 or a control (GL2, drosophila) for 48 hours, then measured cell survival using a luminescent cell viability assay. There was no significant difference in cell viability between the cohort treated with the GL2-targeting and the cohort treated with KIF15-targeting siRNA pool. In cells treated with TPX2-targeting siRNA pools, cell viability was decreased significantly in each cell line. Cell viability was reduced in A2780, CP70, OVCAR-5 and SKOV3 by 74%, 86%, 77% and 75%, respectively, with all calculated p-values less than 0.05. Next, the protocol subjected each cell line to treatment with either siKIF15, siTPX2 or siGL2 (control) for 24 hours, then treated the cells with increasing concentrations of ispinesib as described above. After 72 hours, cell viability was measured and concentration-response curves for each siRNA treatment were plotted and compared. As expected, cells transfected/treated with siTPX2/ispinesib exhibited significant reductions in cell viability compared with ispinesib treatment alone. Importantly, treatment with siKIF15/ispinesib also reduced cell viability as compared with ispinesib alone.

A High Throughput Screen Identifies candidate compounds to inhibit the KIF15-TPX2 Protein-Protein Interaction

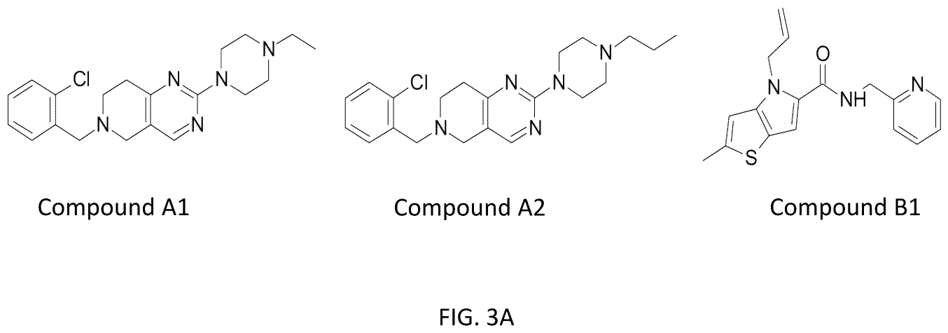

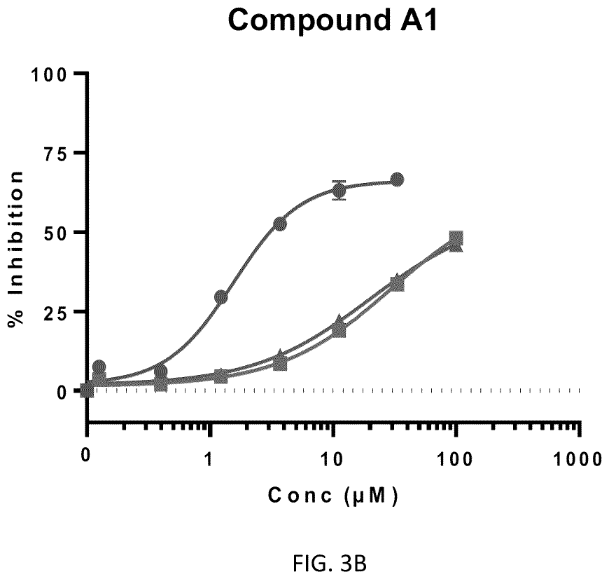

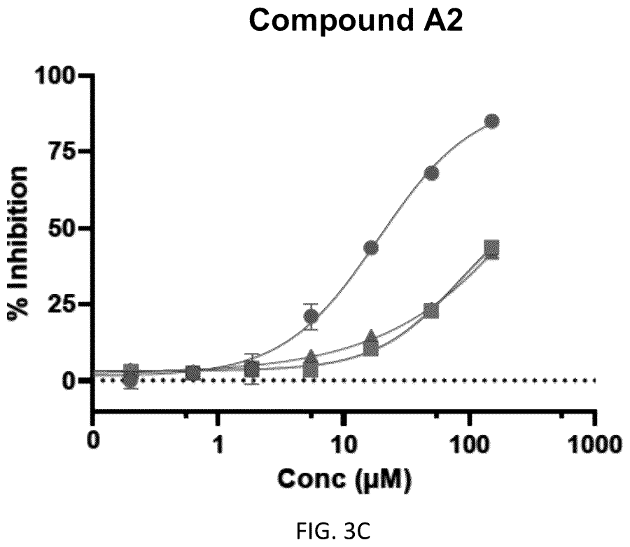

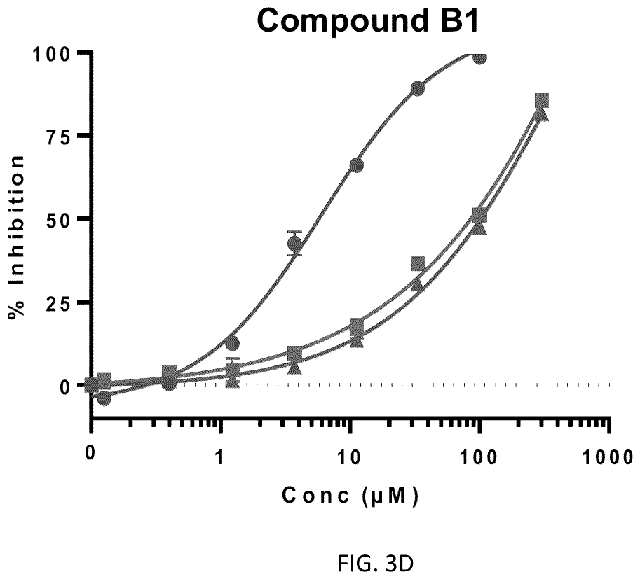

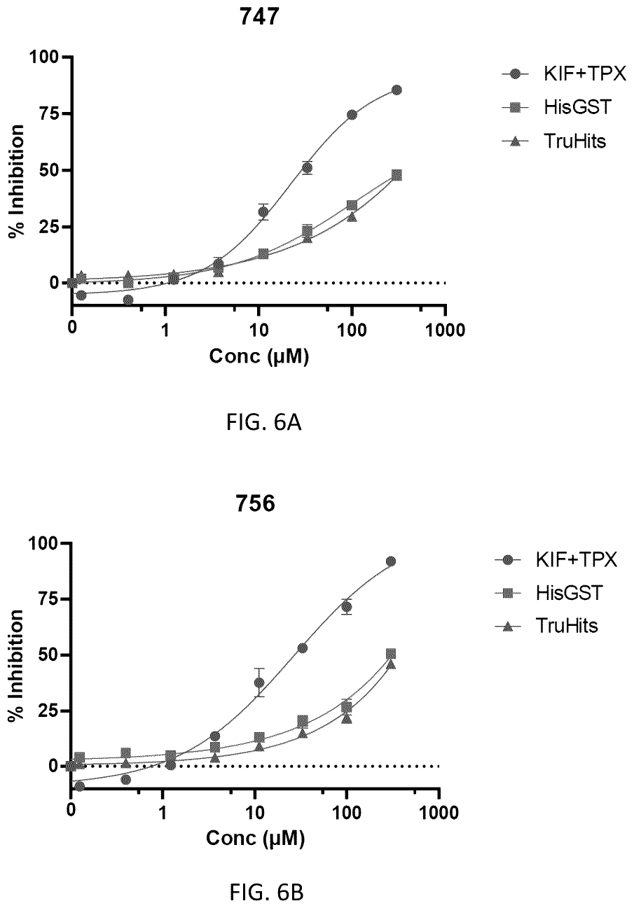

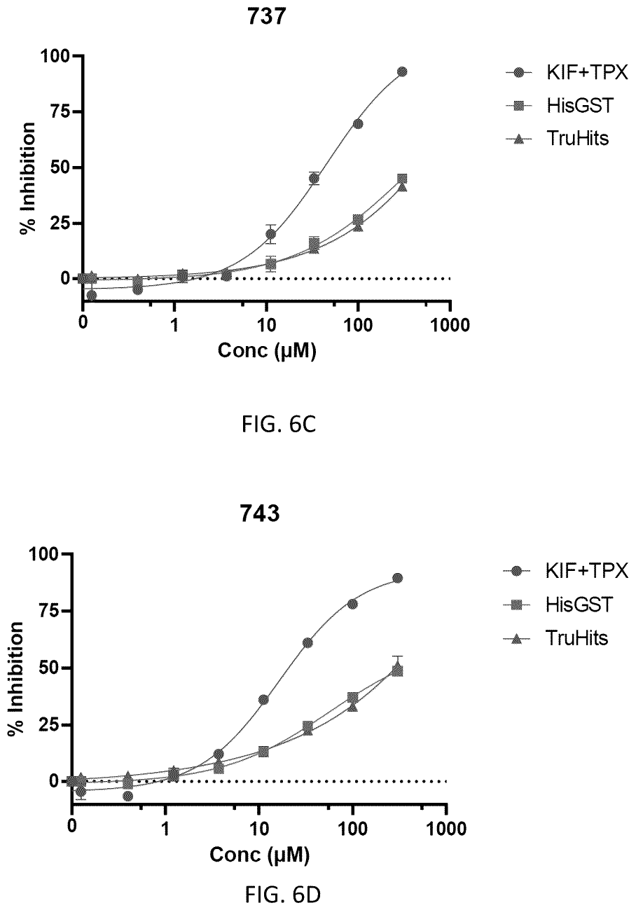

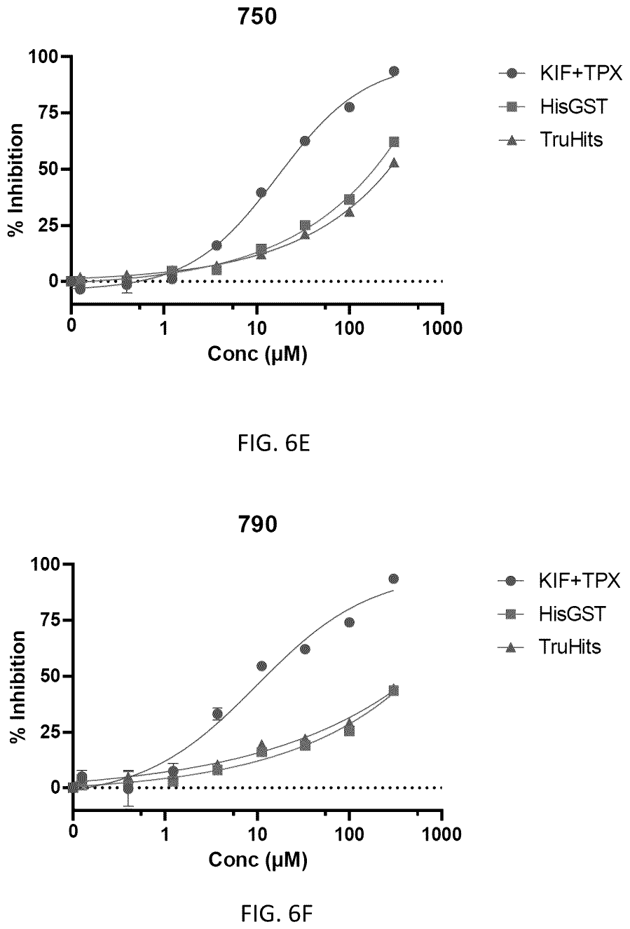

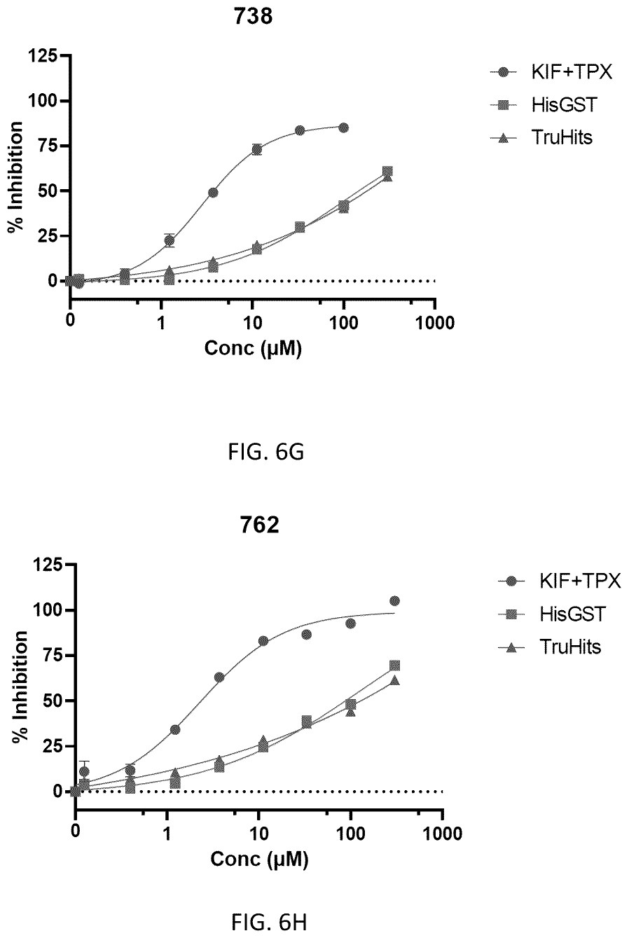

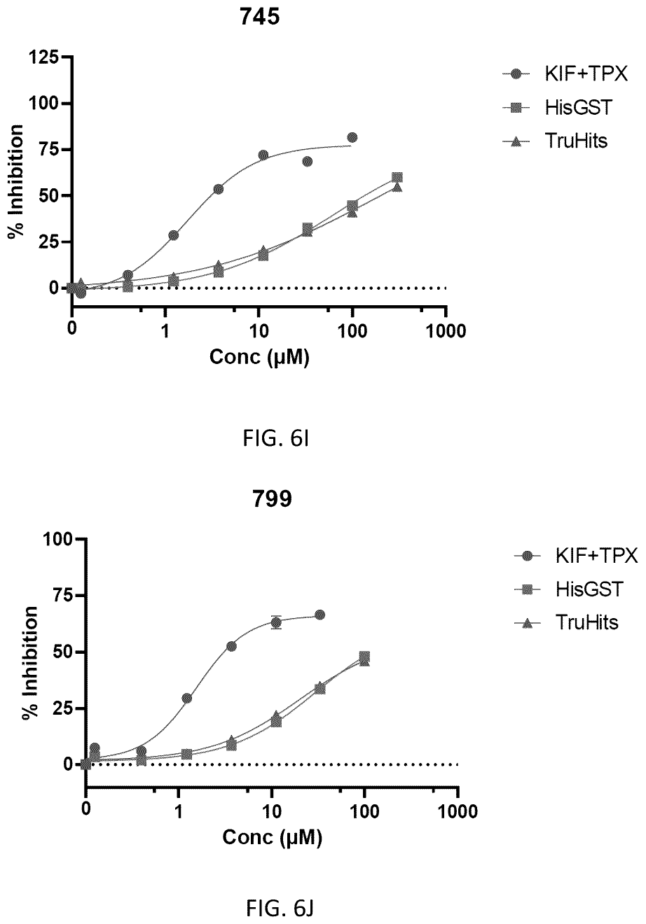

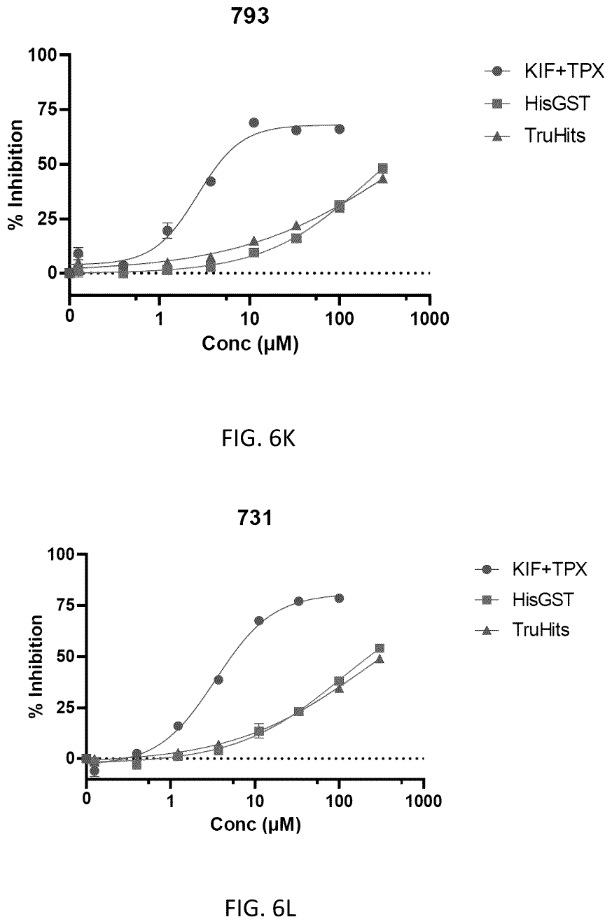

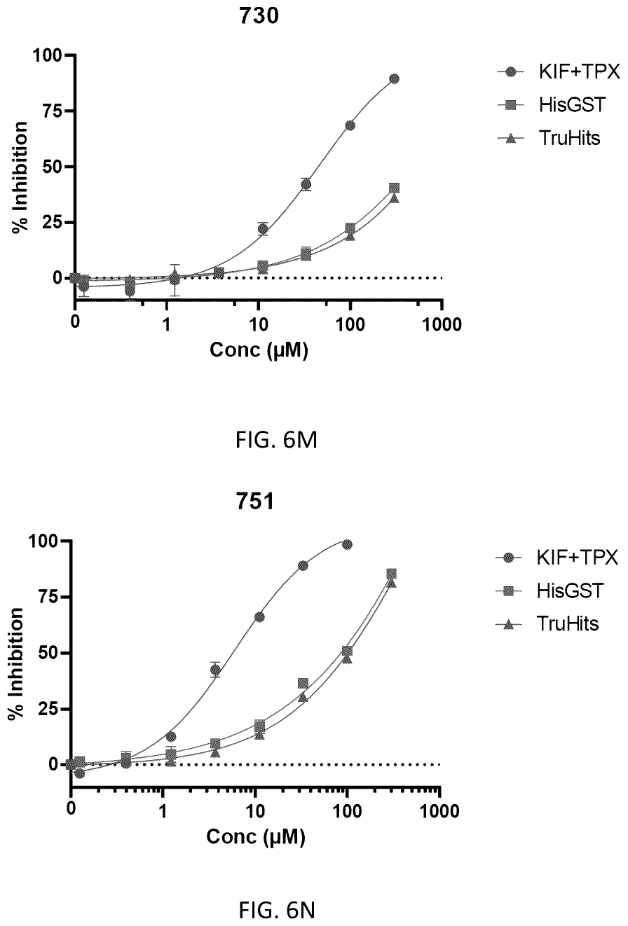

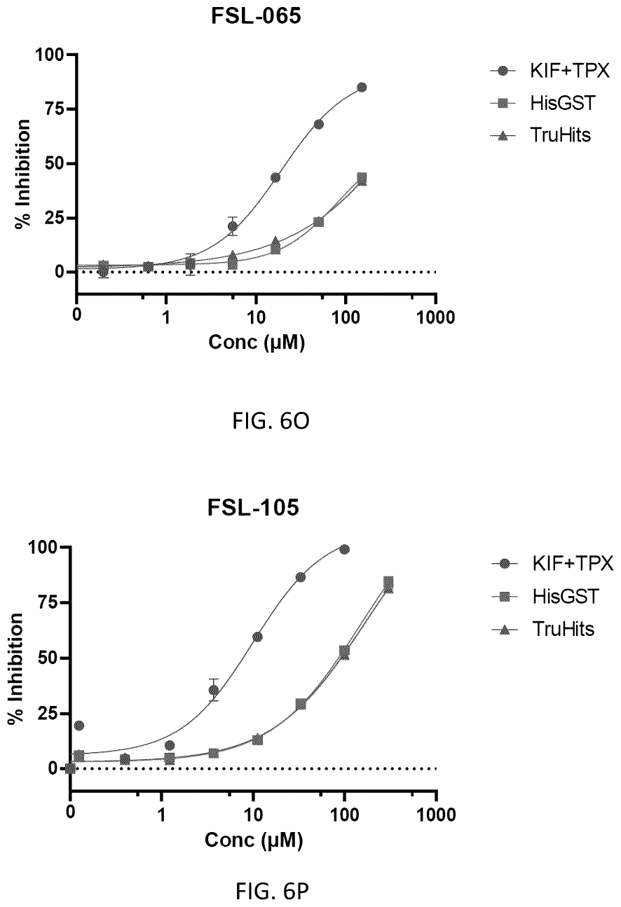

The high throughput screen identified 3 molecules, shown as Compound A1, Compound A2, and Compound B1 (which are shown in FIG. 3A. FIGS. 3B, 3C, and 3D), show the inhibition percentage versus concentration of these respective compounds. In FIGS. 3B, 3C, and 3D, the circles are for KIF+TPX, the triangles are for TruHits, and the squares are for HisGST. Additionally, FIGS. 6A-6P show the percent inhibition for the compounds, which are described further in Table 1. Table 1 shows the HTS code for each of the compounds, which HTS code is included in FIGS. 6A-6P to designate the compound. Table 1 also shows the KIF15/TPX2 AlphaScreen IC50, His-GST AlphaScrean IC5, TruHits IC50, and the NMR versus GST-KIF15 hits.

The protocol designed peptides corresponding to the appropriate domains of KIF15 (amino acids 1149-1388, GST-tagged) and TPX2 (amino acids 346-747, His-tagged), for use in the AlphaScreen assay. Following several assay optimization steps (e.g., buffer selection, protein titration, TruHits assay validation), the protocol demonstrated that KIF15 binds TPX2 specifically, producing an Alpha signal more than 30-fold greater than background. To confirm the observation that KIF15 and TPX2 exhibit specific binding in our HTS assay, the protocol designed two unique peptides that mimic the interacting coiled-coil structures of each protein. Each mimetic peptide exhibited >90% inhibition in the AlphaScreen assay and a concentration dependent inhibition of TPX2-KIF15 binding.

Next, the protocol screened a total of 191,852 compounds from the KU-HTS library. Analysis identified 584 hits that inhibited the primary AlphaScreen TPX2-KIF15 interaction to greater than three standard deviations plus the plate inhibition-median. The 584 hits were tested for assay interference in the AlphaScreen counter-screen using His-GST fusion protein. The 467 compounds that did not show inhibition in the His-GST AlphaScreen counterscreen were tested for reconfirmation in a concentration-response in a TPX2-KIF15 AlphaScreen assay. Of the 467 hits, 399 inhibited the TPX2-KIF15 interaction in a dose-response manner. Refinement was conducted until identifying the compounds provided herein.

Biophysical Assays Confirm Compounds that Bind to KIF15

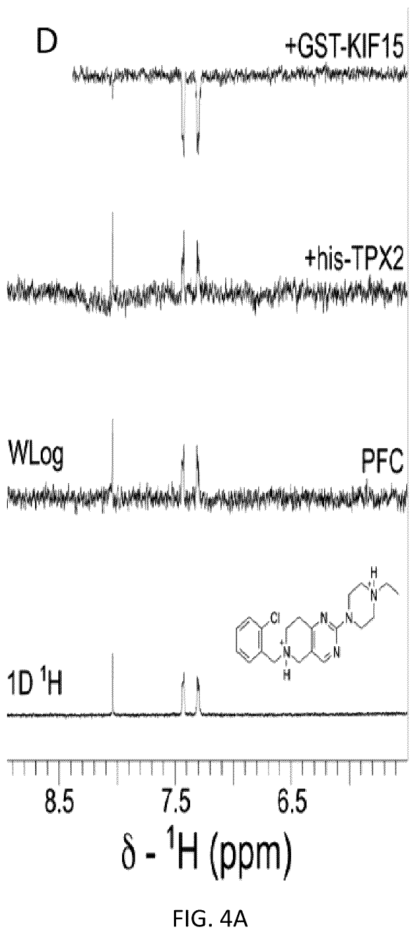

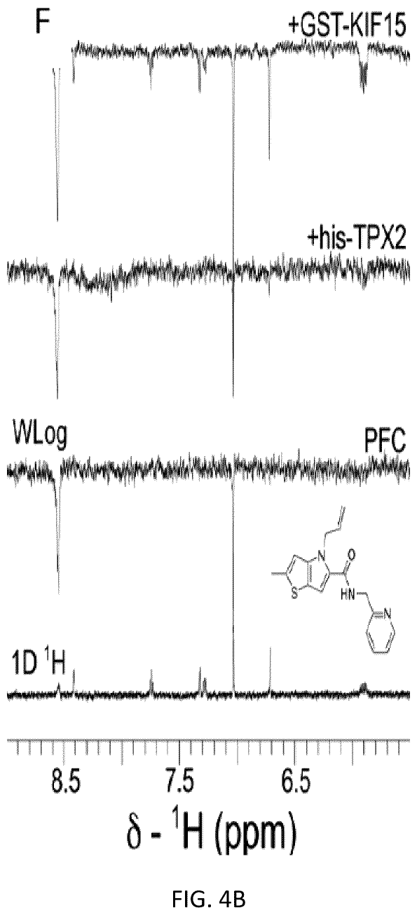

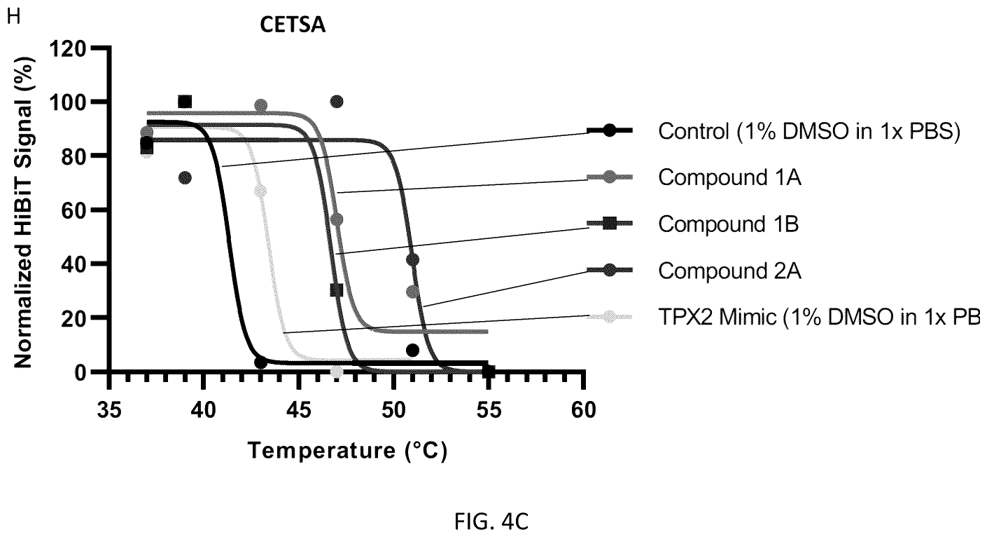

WaterLOGSY NMR Spectra of Compounds A1 and B2 are provided. FIG. 4A shows the NMR spectra of Compound A1, and FIG. 4B shows the NMR spectra of Compound B1. The inversion of the sign of non-exchangeable ligand proton signals in the WaterLOGSY in the presence 1:150 GST-KIF151149-1388 (top) relative to the 1D 1H NMR spectrum (bottom) indicates binding, not observed in the presence 1:150 His-TPX2346-747 (second from top) or the Protein Free Control (third from top).

To verify that the three putative binders of KIF15 identified by NMR spectroscopy against KIF15 fragments, a cellular thermal shift assay was performed. All three compounds at 100 μM stabilized KIF15 when thermally challenged to a greater extent than the TPX2 mimetic peptide used as a positive control (Δ5.7° C. for compound A1, Δ5.3° C. for compound A2, and Δ9.6° C. for compound B1, Δ2.1° C. for TPX2 mimetic peptide).

The HiBiT Split Luciferase based protein detection method utilized for screening of candidate compounds for physical binding to KIF15. FIG. 4C shows a graph with melt curves of KIF15-HiBiT thermally challenged for 5 minutes in the presences on 100 μM of Compound A1, A2, B1 or TPX2 mimetic peptide. The increase in stability of the protein indicates ligand engagement by stabilizing the protein in a natured conformation.

Compounds A1, A2, and B1 Exhibit Synergy with Ispinesib in the Inhibition of Growth of Ovarian Cancer Cell Lines

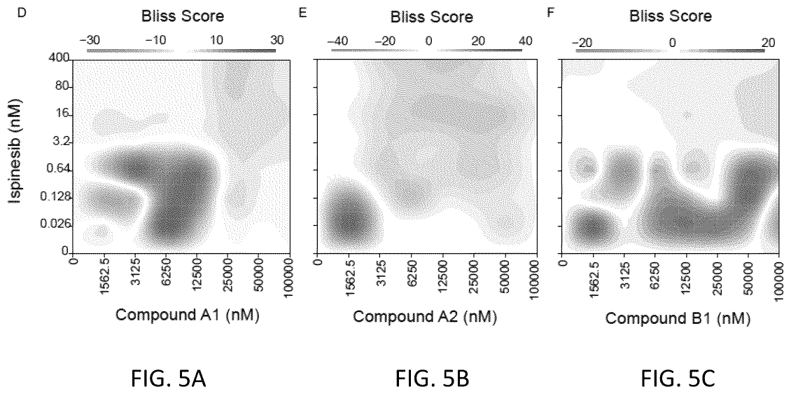

The protocol tested all 166 samples of compound hits for complete screening for synergism in KIF11-inhibited cells EOC cell lines (A2780, OVCAR5, and SKOV3) (FIGS. 5A-5C). As predicted, the data showed synergistic activity for all 3 compound hits confirmed to bind to KIF15 by STD-NMR and/or WaterLOGSY.

The viability of A2780 were assessed when treated with ispinesib and putative KIF15-TPX2 PPI inhibitor Compound A1, Compound A2, and Compound B1. The combination of ispinesib and KIF15 were analyzed for synergism by Bliss score. Bliss score analyzes the statistical independence of the two treatments. A positive score indicates synergism, a negative score indicates antagonism and a score of zero indicates a purely additive effect of the combination therapy. FIG. 5A shows the Bliss score for Compound A1. FIG. 5B shows the Bliss score for Compound A2. FIG. 5C shows the Bliss score for Compound B1.

Various cocktails of compounds were prepared to test for interactions. 100 μL DMSO-d6 was added to 3 mg of material. 26 cocktails of 6 compounds were prepared and 3 cocktails of 7 compounds were prepared. Then, 3 μM protein is added to 300 μM of the cocktails in 10% D₂O buffer. Concentration of compounds is 10 mM each.

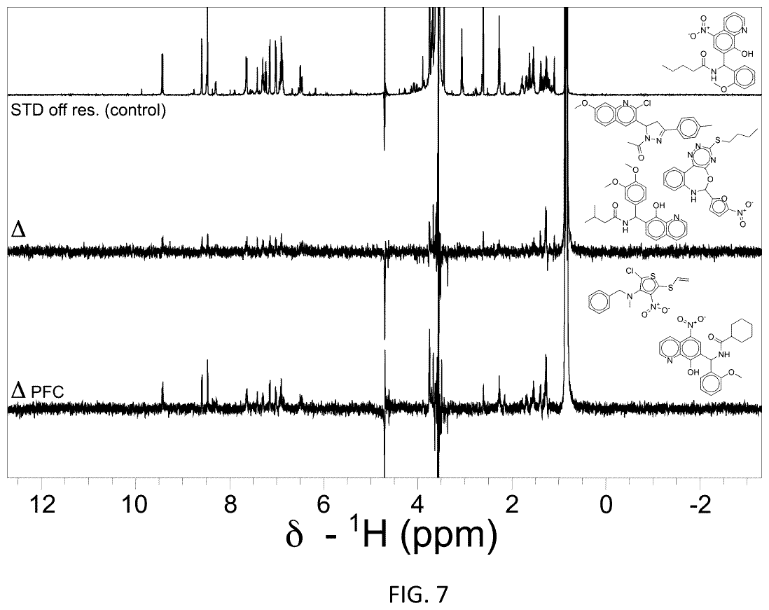

FIG. 7 shows the data for a screen of cocktail #4, which shows hits.

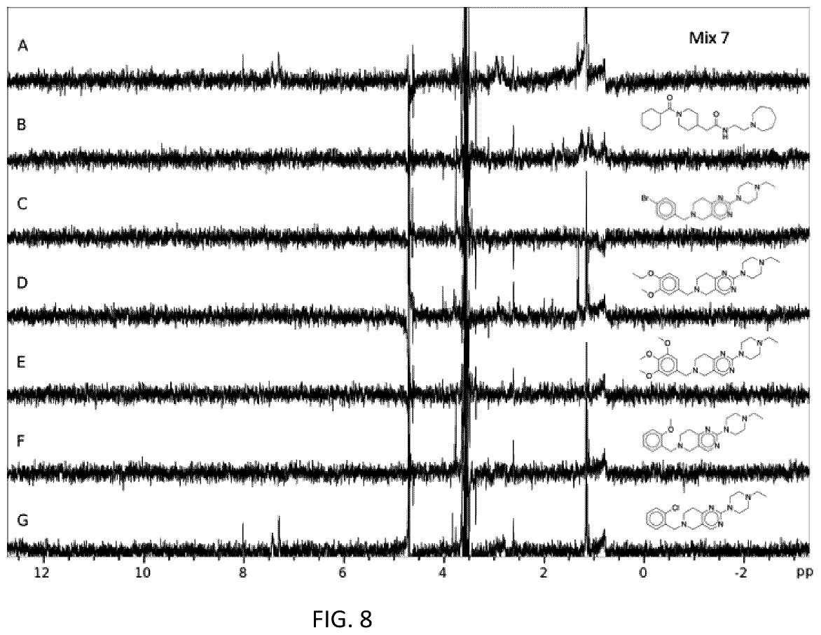

FIG. 8 shows the data for a screen of cocktail #7, which shows hits. The dereplicate of the single compounds also shows the hits by screening the individual compounds.

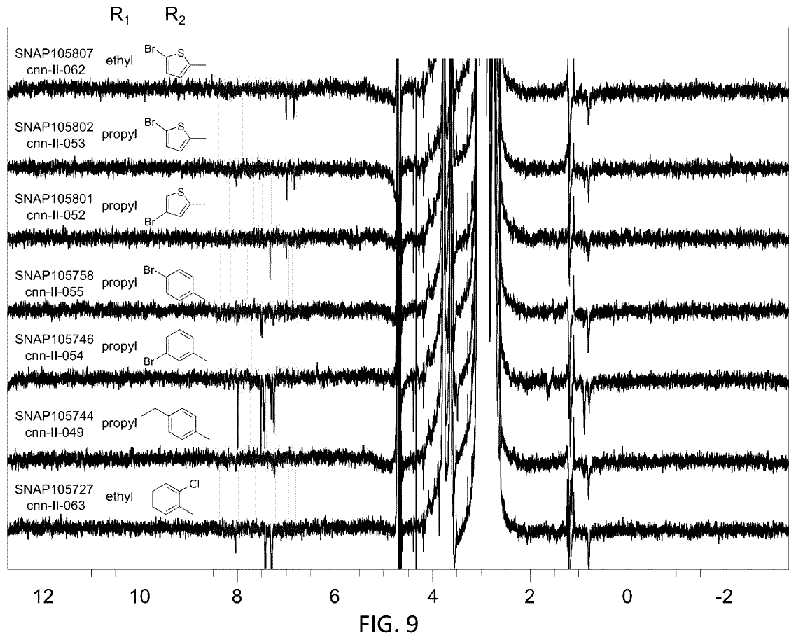

Additionally, a structure-activity relationship was investigated using the WLOG. FIG. 9 shows the structure-activity relationship using WLOG.

### Experiments

The protocol employed a high-throughput biochemical AlphaScreen primary screen (˜200K compounds), associated counter-screens and follow-on biophysical and cell-based counter-screen and orthogonal assays to validate small-molecule compound hits representing two structurally distinct chemotypes that inhibit the KIF15/TPX2 protein/protein interaction (PPI). Our proposed studies to optimize these two KIF15i compound-hit chemotypes into lead candidates suitable for use in in vivo proof-of-concept studies in combination with known KIF11 inhibitors to explore our hypothesis that targeting the KIF11/KIF15/TPX2 axis represents a tractable approach to improved treatments for EWS and OC are innovative, in that, the hundreds-of-thousands of PPI that are central to biological processes represent a significant and clinically underrepresented class of therapeutic targets⁹⁰.

Structurally distinct small-molecule fragments can be discovered through independent Fragment Based Ligand Discovery studies that bind to the KIF15 motor domain into KIF15/TPX2 PPI-selective ATP-competitive inhibitors, based on our experience with PPI-selective ATP-competitive inhibitors of the ATPase p97^{91, 92}.

The screening can include a RNAi-based screen which identified KIF11 (Eg5) as a putative vulnerability in a molecularly and histologically diverse panel of epithelial ovarian cancer (EOC) cell lines^{4, 93.}

To identify compounds that inhibit KIF15-mediated bipolar spindle formation, a process that is dependent on KIF15 binding to TPX2, the protocol designed a high-throughput screen using AlphaScreen Technology. Previous studies predicted that KIF15-TPX2 binding is mediated through a C-terminal coiled-coil domain on KIF15 (amino acids 1149-1388; amino acids 1359-1380) that binds the C-terminal region of TPX2 (amino acids 475-715; amino acids 680-747)^{50, 52, 57, 58}. Based on these observations, the protocol designed peptides corresponding to the appropriate domains of TPX2 (amino acids 346-747, His-tagged), and KIF15 (amino acids 1149-1388, GST-tagged) for use in the AlphaScreen assay. Following several assay optimization steps (e.g., buffer selections, protein titration, TruHits counter-screen assay validation), the data demonstrated that KIF15 binds TPX2 specifically, producing a signal more than 30-fold greater than background. To confirm our observation that GST-KIF15₁₁₄₉₋₁₃₈₈ and His-TPX2₃₄₆₋₇₄₇ exhibit specific binding in our HTS assay, the protocol designed two unique short peptides that mimic the interacting coiled-coil structures of each protein. Each mimetic peptide exhibited inhibition in the AlphaScreen assay and a concentration-dependent inhibition of KIF15-TPX2 binding.

Next, the protocol screened a total of 191,852 compounds from the KU-HTS small-molecule compound collection. The protocol identified 584 hits that inhibited the primary AlphaScreen TPX2-KIF15 interaction to greater than three standard deviations plus the plate inhibition-median. The 584 hits were tested for assay interference in the AlphaScreen assay counter-screen using His-GST fusion protein eliminating 117 compounds⁹⁹. The remaining 467 compounds that did not show inhibition in the His-GST AlphaScreen counter-screen were tested for reconfirmation in a concentration-response in TPX2-KIF15 AlphaScreen assay. Of the 467 hits, 399 inhibited the KIF15-TPX2 interaction in a concentration-dependent manner (FIGS. 3A-3D), rendering them candidates for follow-on validation in orthogonal and counter-screen assays. Informed by computational and medicinal chemistry assessment¹⁰⁰, the protocol selected a representative subset of approximately 200 of the 399 compound hits for additional characterization. Samples for 177 compounds from the subset of approximately 200 representative compounds were available for purchase. Samples of these 177 compounds were purchased for structure verification by NMR and further characterization. There was enough compound sample procured to complete follow-on screening for 166 of the 177 compounds. The confirmatory AlphaScreen assays results for our two validated compound chemotypes/hits are shown in the FIGS. 3A-3D.

The 166 compound hit samples were further characterized for KIF15 or TPX2 binding using biophysical saturation transfer difference nuclear magnetic resonance (STD-NMR) and WaterLOGSY assays. The 166 compound samples were screened by STD-NMR for evidence of binding to GST-KIF15 or His-TPX2. In our assessment, no hits bound to His-TPX2. The protocol counter-screened potential hits against a His-GST peptide⁹⁹ and for aggregation using protein-free controls. Ultimately, three compound hits representing two compound chemotypes were validated to bind KIF15 by WaterLOGSY (FIGS. 4A-4B).

These three compound hits, referred to as Compounds A1, A2 and B1 (FIG. 3A) were screened for target engagement in CETSA. In short, a split luciferase tag known as HiBiT (Promega, Madison, WI) was fused to the KIF15 protein in a transient expression vector. Soluble protein can be easily quantified by adding the LgBiT subunit of nano luciferase. A stabilizing shift in the melting temperature of the protein indicates the protein is being stabilized by the engaged small molecule indicating direct binding of the small molecule to its target. At a concentration 100 μM, compounds A1 and B1 exhibited a significant shift in thermostability.

### Definitions

By “substituted” as in “substituted alkyl,” “substituted aryl,” and the like, as alluded to in some of the definitions provided herein, is meant that in the alkyl, aryl, or other moiety, at least one hydrogen atom bound to a carbon (or other) atom is replaced with one or more non-hydrogen substituents. In addition, the aforementioned functional groups may, if a particular group permits, be further substituted with one or more additional functional groups or with one or more hydrocarbyl moieties such as those specifically enumerated above. Analogously, the above-mentioned hydrocarbyl moieties may be further substituted with one or more functional groups or additional hydrocarbyl moieties such as those specifically enumerated. When the term “substituted” appears prior to a list of possible substituted groups, it is intended that the term apply to every member of that group. For example, the phrase “substituted alkyl, alkenyl, and aryl” is to be interpreted as “substituted alkyl, substituted alkenyl, and substituted aryl.” Analogously, when the term “heteroatom-containing” appears prior to a list of possible heteroatom-containing groups, it is intended that the term apply to every member of that group. For example, the phrase “heteroatom-containing alkyl, alkenyl, and aryl” is to be interpreted as “heteroatom-containing alkyl, heteroatom-containing alkenyl, and heteroatom-containing aryl.”

As used herein, “optionally substituted” or other similar term indicates that a chemical structure may be optionally substituted with a substituent group, such as defined herein. That is, when a chemical structure includes an atom that is optionally substituted, the atom may or may not include the optional substituent group, and thereby the chemical structure may be considered to be substituted when having a substituent on the atom or unsubstituted when omitting a substituent from the atom. A substituted group, referred to as a “substituent” or “substituent group”, can be coupled (e.g., covalently) to a previously unsubstituted parent structure, wherein one or more hydrogens atoms (or other substituent groups) on the parent structure have been independently replaced by one or more of the substituents. The substituent is a chemical moiety that is added to a base chemical structure, such as a chemical scaffold. As such, a substituted chemical structure may have one or more substituent groups on the parent structure, such as by each substituent group being coupled to an atom of the parent structure. The substituent groups that can be coupled to the parent structure can be any possible substituent group. In examples of the present technology, the substituent groups (e.g., R groups) can be independently selected from an alkyl, —O-alkyl (e.g. —OCH₃, —OC₂H₅, —OC₃H₇, —OC₄H₉, etc.), —S-alkyl (e.g., —SCH₃, —SC₂H₅, —SC₃H₇, —SC₄H₉, etc.), —NR′R″, —OH, —SH, —CN, —NO₂, or a halogen, wherein R′ and R″ are independently H or an optionally substituted alkyl. Wherever a substituent is described as “optionally substituted,” that substituent can also be optionally substituted with the above substituents.

The term “alkyl” or “aliphatic” as used herein refers to a branched or unbranched saturated hydrocarbon group typically although not necessarily containing 1 to about 24 carbon atoms, such as methyl, ethyl, n-propyl, isopropyl, n-butyl, isobutyl, t-butyl, octyl, decyl, and the like, as well as cycloalkyl groups such as cyclopentyl, cyclohexyl, and the like. Generally, although again not necessarily, alkyl groups herein contain 1 to about 18 carbon atoms, or 1 to about 12 carbon atoms. The term “lower alkyl” intends an alkyl group of 1 to 6 carbon atoms. Substituents identified as “C₁-C₆ alkyl” or “lower alkyl” contains 1 to 3 carbon atoms, and such substituents contain 1 or 2 carbon atoms (i.e., methyl and ethyl). “Substituted alkyl” refers to alkyl substituted with one or more substituent groups, and the terms “heteroatom-containing alkyl” and “heteroalkyl” refer to alkyl in which at least one carbon atom is replaced with a heteroatom, as described in further detail infra. If not otherwise indicated, the terms “alkyl” and “lower alkyl” include linear, branched, cyclic, unsubstituted, substituted, and/or heteroatom-containing alkyl or lower alkyl, respectively.

The term “cycloalkyl”, as used herein, refers to a monocyclic C₃-C₈ hydrocarbon that is completely saturated or that contains one or more units of unsaturation, but which is not aromatic, that has a single point of attachment to the rest of the molecule. Exemplary groups include cyclopropyl, cyclobutyl, cyclopentyl, cyclohexyl, cycloheptyl, cycooctyl, cyclodecyl, cyclododecyl and adamantyl.

The terms “alkenyl” as used herein refers to a linear, branched, or cyclic hydrocarbon group of 2 to about 24 carbon atoms containing at least one double bond, such as ethenyl, n-propenyl, isopropenyl, n-butenyl, isobutenyl, octenyl, decenyl, tetradecenyl, hexadecenyl, eicosenyl, tetracosenyl, and the like. Generally, although again not necessarily, alkenyl groups herein contain 2 to about 18 carbon atoms, or 2 to 12 carbon atoms. The term “lower alkenyl” intends an alkenyl group of 2 to 6 carbon atoms, and the specific term “cycloalkenyl” intends a cyclic alkenyl group or having 5 to 8 carbon atoms. The term “substituted alkenyl” refers to alkenyl substituted with one or more substituent groups, and the terms “heteroatom-containing alkenyl” and “heteroalkenyl” refer to alkenyl in which at least one carbon atom is replaced with a heteroatom. If not otherwise indicated, the terms “alkenyl” and “lower alkenyl” include linear, branched, cyclic, unsubstituted, substituted, and/or heteroatom-containing alkenyl and lower alkenyl, respectively.

The term “alkynyl” as used herein refers to a linear or branched hydrocarbon group of 2 to 24 carbon atoms containing at least one triple bond, such as ethynyl, n-propynyl, and the like. Generally, although again not necessarily, alkynyl groups herein contain 2 to about 18 carbon atoms, or 2 to 12 carbon atoms. The term “lower alkynyl” intends an alkynyl group of 2 to 6 carbon atoms. The term “substituted alkynyl” refers to alkynyl substituted with one or more substituent groups, and the terms “heteroatom-containing alkynyl” and “heteroalkynyl” refer to alkynyl in which at least one carbon atom is replaced with a heteroatom. If not otherwise indicated, the terms “alkynyl” and “lower alkynyl” include linear, branched, unsubstituted, substituted, and/or heteroatom-containing alkynyl and lower alkynyl, respectively.

The term “alkoxy” as used herein intends an alkyl group bound through a single, terminal ether linkage; that is, an “alkoxy” group may be represented as O-alkyl where alkyl is as defined above. A “lower alkoxy” group intends an alkoxy group containing 1 to 6 carbon atoms, and includes, for example, methoxy, ethoxy, n-propoxy, isopropoxy, t-butyloxy, etc. Substituents identified as “C₁-C₆ alkoxy” or “lower alkoxy” herein contain 1 to 3 carbon atoms, and such substituents contain 1 or 2 carbon atoms (i.e., methoxy and ethoxy).

The term “aryl” as used herein, and unless otherwise specified, refers to an aromatic substituent containing a single aromatic ring or multiple aromatic rings that are fused together, directly linked, or indirectly linked (such that the different aromatic rings are bound to a common group such as a methylene or ethylene moiety). Examples of aryl groups contain 5 to 20 carbon atoms, and aryl groups contain 5 to 14 carbon atoms. Exemplary aryl groups contain one aromatic ring or two fused or linked aromatic rings, e.g., phenyl, naphthyl, biphenyl, diphenylether, diphenylamine, benzophenone, and the like. “Substituted aryl” refers to an aryl moiety substituted with one or more substituent groups, and the terms “heteroatom-containing aryl” and “heteroaryl” refer to aryl substituent, in which at least one carbon atom is replaced with a heteroatom, as will be described in further detail infra. If not otherwise indicated, the term “aryl” includes unsubstituted, substituted, and/or heteroatom-containing aromatic substituents.

The terms “heteroaryl” used alone or as part of a larger moiety, e.g., “heteroaralkyl”, or “heteroaralkoxy”, refer to groups having 5 to 10 ring atoms, preferably 5, 6, or 9 ring atoms; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl, indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one.

The term “heteroaralkyl” refers to an alkyl group substituted by a heteroaryl, bonded with alkyl and heteroaryl portions.

The term “aryloxy” as used herein refers to an aryl group bound through a single, terminal ether linkage, wherein “aryl” is as defined above. An “aryloxy” group may be represented as —O-aryl where aryl is as defined above. Examples of aryloxy groups contain 5 to 20 carbon atoms, and aryloxy groups contain 5 to 14 carbon atoms. Examples of aryloxy groups include, without limitation, phenoxy, o-halo-phenoxy, m-halo-phenoxy, p-halo-phenoxy, o-methoxy-phenoxy, m-methoxy-phenoxy, p-methoxy-phenoxy, 2,4-dimethoxy-phenoxy, 3,4,5-trimethoxy-phenoxy, and the like.

The term “alkaryl” refers to an aryl group with an alkyl substituent, and the term “aralkyl” refers to an alkyl group with an aryl substituent, wherein “aryl” and “alkyl” are as defined above. Examples of aralkyl groups contain 6 to 24 carbon atoms, and aralkyl groups contain 6 to 16 carbon atoms. Examples of aralkyl groups include, without limitation, benzyl, 2-phenyl-ethyl, 3-phenyl-propyl, 4-phenyl-butyl, 5-phenyl-pentyl, 4-phenylcyclohexyl, 4-benzylcyclohexyl, 4-phenylcyclohexylmethyl, 4-benzylcyclohexylmethyl, and the like. Alkaryl groups include, for example, p-methylphenyl, 2,4-dimethylphenyl, p-cyclohexylphenyl, 2,7-dimethyinaphthyl, 7-cyclooctylnaphthyl, 3-ethyl-cyclopenta-1,4-diene, and the like.

The term “cyclic” refers to alicyclic or aromatic substituents that may or may not be substituted and/or heteroatom containing, and that may be monocyclic, bicyclic, or polycyclic.

The terms “halo” and “halogen” are used in the conventional sense to refer to a chloro, bromo, and fluoro or iodo substituent.

The term “heteroatom-containing” as in a “heteroatom-containing alkyl group” (also termed a “heteroalkyl” group) or a “heteroatom-containing aryl group” (also termed a “heteroaryl” group) refers to a molecule, linkage or substituent in which one or more carbon atoms are replaced with an atom other than carbon, e.g., nitrogen, oxygen, sulfur, phosphorus or silicon, typically nitrogen, oxygen or sulfur. Similarly, the term “heteroalkyl” refers to an alkyl substituent that is heteroatom-containing, the term “heterocyclic” refers to a cyclic substituent that is heteroatom-containing, the terms “heteroaryl” and heteroaromatic” respectively refer to “aryl” and “aromatic” substituents that are heteroatom-containing, and the like. Examples of heteroalkyl groups include alkoxyaryl, alkylsulfanyl-substituted alkyl, N-alkylated amino alkyl, and the like. Examples of heteroaryl substituents include pyrrolyl, pyrrolidinyl, pyridinyl, quinolinyl, indolyl, pyrimidinyl, imidazolyl, 1,2,4-triazolyl, tetrazolyl, etc., and examples of heteroatom-containing alicyclic groups are pyrrolidino, morpholino, piperazino, piperidino, etc.

The term “hydrocarbyl” refers to univalent hydrocarbyl radicals containing 1 to about 30 carbon atoms, or 1 to about 24 carbon atoms, or 1 to about 18 carbon atoms, or about 1 to 12 carbon atoms, including linear, branched, cyclic, saturated, and unsaturated species, such as alkyl groups, alkenyl groups, aryl groups, and the like. “Substituted hydrocarbyl” refers to hydrocarbyl substituted with one or more substituent groups, and the term “heteroatom-containing hydrocarbyl” refers to hydrocarbyl in which at least one carbon atom is replaced with a heteroatom. Unless otherwise indicated, the term “hydrocarbyl” is to be interpreted as including substituted and/or heteroatom-containing hydrocarbyl moieties.

A “prodrug” is a compound which is converted to a therapeutically active compound after administration, and the term should be interpreted as broadly herein as is generally understood in the art. While not intending to limit the scope of the invention, conversion may occur by hydrolysis of an ester group or some other biologically labile group. Generally, but not necessarily, a prodrug is inactive or less active than the therapeutically active compound to which it is converted. Ester prodrugs of the compounds disclosed herein are specifically contemplated. An ester may be derived from a carboxylic acid of an R group, or an ester may be derived from a carboxylic acid functional group on another part of the molecule. Any atom may be linked to a prodrug, however, the R groups are particularly useful to be conjugated through an ester to the prodrug moiety. While not intending to be limiting, an ester may be an alkyl ester, an aryl ester, or a heteroaryl ester. The prodrug may also include a phosphate, amino acid, carbonate, carbamate, or lipid, which can be cleaved from the subject compounds described herein through a cleavable linker.

The term “subject”, as used herein, means an animal, preferably a cell, biological tissue, or animal, preferably mammal, and most preferably a human.

The term “pharmaceutically acceptable carrier or pharmaceutically acceptable vehicle” refers to a non-toxic carrier, adjuvant, or vehicle that does not substantially alter the pharmacological activity of the compound with which it is formulated. Pharmaceutically acceptable carriers or vehicles that are used in the compositions of this invention include, but are not limited to, lecithin, glycine, sorbic acid, potassium sorbate, sodium chloride, zinc salts, colloidal silica, magnesium trisilicate, polyvinyl pyrrolidone, cellulose-based substances, polyethylene glycol, buffer substances such as cytric acid and phosphates.

In particular, the compounds described herein can be administered orally, parenterally, by inhalation spray, topically, rectally, nasally, buccally, vaginally or via an implanted reservoir.

The term “therapeutically effective amount”, as used herein, refers to a dosage and duration of administration which is commonly known in the art and recognized and utilized by the medical community. Such an amount will vary depending on the particular agent(s) administered, the size and/or condition of the subject receiving treatment or other medical factors determined by the administering physician.

In certain embodiments, the cancers include, but are not limited to cancer of the breast, bladder, bone, brain, central and peripheral nervous system, colon, sarcoma, ovary, pancreas, prostate, rectum, renal, small intestine, soft tissue, testis, stomach, skin, ureter, vagina and vulva; inherited cancers, retinomblastoma, Wilms tumor, leukemia, lymphoma, non-Hodgkins disease, chronic and acute myeloid leukaemia, acute lymphoblastic leukemia, Hodgkin's disease, multiple myeloma, and T-cell lymphoma, myelodysplastic syndrome, and AIDS related cancer type diseases.

All other chemistry terms are defined as known in the art.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.
1. Pessetto Z Y, Chen B, Alturkmani H, Hyter S, Flynn C A, Baltezor M, Ma Y, Rosenthal H G, Neville K A, Weir S J, Butte A J, Godwin A K. In silico and in vitro drug screening identifies new therapeutic approaches for Ewing sarcoma. Oncotarget. 2017; 8(3):4079-95. Epub 2016/11/20. doi: 10.18632/oncotarget.13385. PubMed PMID: 27863422; PMCID: PMC5354814.
2. Ma Y, Baltezor M, Rajewski L, Crow J, Samuel G, Staggs V S, Chastain K M, Toretsky J A, Weir S J, Godwin A K. Targeted inhibition of histone deacetylase leads to suppression of Ewing sarcoma tumor growth through an unappreciated EWS-FLI1/HDAC3/HSP90 signaling axis. J Mol Med (Berl). 2019; 97(7):957-72. Epub 2019/04/27. doi: 10.1007/s00109-019-01782-0. PubMed PMID: 31025088; PMCID: PMC6584050.
3. Pathak H B, Zhou Y, Sethi G, Hirst J, Schilder R J, Golemis E A, Godwin A K. A Synthetic Lethality Screen Using a Focused siRNA Library to Identify Sensitizers to Dasatinib Therapy for the Treatment of Epithelial Ovarian Cancer. PLoS One. 2015; 10(12):e0144126. Epub 20151204. doi: 10.1371/journal.pone.0144126. PubMed PMID: 26637171; PMCID: PMC4670180.
4. Sethi G, Pathak H B, Zhang H, Zhou Y, Einarson M B, Vathipadiekal V, Gunewardena S, Birrer M J, Godwin A K. An RNA interference lethality screen of the human druggable genome to identify molecular vulnerabilities in epithelial ovarian cancer. PloS one. 2012; 7(10):e47086. Epub 2012/10/12. doi: 10.1371/journal.pone.0047086. PubMed PMID: 23056589; PMCID: PubMed PMID: 23056589; PubMed Central PMCID: PMC3467214.
5. Hirst J, Godwin A K. AURKA inhibition mimics BRCAness. Aging (Albany NY). 2017; 9(9):1945-6. doi: 10.18632/aging.101291. PubMed PMID: 28898201; PMCID: PMC5636662.
6. Do T V, Hirst J, Hyter S, Roby K F, Godwin A K. Aurora A kinase regulates non-homologous end-joining and poly(ADP-ribose) polymerase function in ovarian carcinoma cells. Oncotarget. 2017; 8(31):50376-92. Epub 20170705. doi: 10.18632/oncotarget.18970. PubMed PMID: 28881569; PMCID: PMC5584138.
7. Hyter S, Hirst J, Pathak H, Pessetto Z Y, Koestler D C, Raghavan R, Pei D, Godwin A K. Developing a genetic signature to predict drug response in ovarian cancer. Oncotarget. 2018; 9(19):14828-48. Epub 20171226. doi: 10.18632/oncotarget.23663. PubMed PMID: 29599910; PMCID: PMC5871081.
8. Hirst J, Pathak H B, Hyter S, Pessetto Z Y, Ly T, Graw S, Koestler D C, Krieg A J, Roby K F, Godwin A K. Licofelone Enhances the Efficacy of Paclitaxel in Ovarian Cancer by Reversing Drug Resistance and Tumor Stem-like Properties. Cancer Res. 2018; 78(15):4370-85. Epub 20180611. doi: 10.1158/0008-5472.CAN-17-3993. PubMed PMID: 29891506; PMCID: PMC6072598.
9. Garcia-Saez I, Skoufias D A. Eg5 targeting agents: From new anti-mitotic based inhibitor discovery to cancer therapy and resistance. Biochemical pharmacology. 2021; 184:114364. doi: doi.org/10.1016/j.bcp.2020.114364.
10. Surveillance, Epidemiology, and End Results (SEER) Program (cancer.gov) SEER*Stat Database: Mortality—All COD, Aggregated With State, Total U.S. (1969-2019) [Internet]2021.
11. Surveillance, Epidemiology, and End Results (SEER) Program Populations (1969-2020) (cancer.gov/popdata) [Internet]2022.
12. Chang L, Ruiz P, Ito T, Sellers W R. Targeting pan-essential genes in cancer: Challenges and opportunities. Cancer Cell. 2021; 39(4):466-79. Epub 20210114. doi: 10.1016/j.ccell.2020.12.008. PubMed PMID: 33450197; PMCID: PMC8157671.
13. Bergnes G, Conlan M G, Knight S D, Abraham D J. Inhibitors of Kinesin Spindle Protein for the Treatment of Cancer. Burger's Medicinal Chemistry and Drug Discovery: John Wiley & Sons, Inc.; 2003.
14. Cross R A, McAnish A. Prime movers: the mechanochemistry of mitotic kinesins. Nat Rev Mol Cell Biol. 2014; 15(4):257-71. doi: 10.1038/nrm3768; PMCID: PubMed PMID: 24651543.
15. DeBonis S, Skoufias D A, Lebeau L, Lopez R, Robin G, Margolis R L, Wade R H, Kozielski F. In vitro screening for inhibitors of the human mitotic kinesin Eg5 with antimitotic and antitumor activities. Mol Cancer Ther. 2004; 3(9):1079-90. Epub 2004/09/16. PubMed PMID: 15367702; PMCID: PubMed PMID: 15367702.
16. Lad L, Luo L, Carson J D, Wood K W, Hartman J J, Copeland R A, Sakowicz R. Mechanism of Inhibition of Human KSP by Ispinesib. Biochemistry. 2008; 47(11):3576-85. doi: 10.1021/bi702061g; PMCID: PubMed PMID: 18290633.
17. Wojcik E J, Buckley R S, Richard J, Liu L, Huckaba T M, Kim S. Kinesin-5: cross-bridging mechanism to targeted clinical therapy. Gene. 2013; 531(2):133-49. Epub 2013/08/21. doi: 10.1016/j.gene.2013.08.004. PubMed PMID: 23954229; PMCID: PubMed PMID: 23954229; PubMed Central PMCID: PMC3801170.
18. Rath O, Kozielski F. Kinesins and cancer. Nat Rev Cancer. 2012; 12(8):527-39. Epub 20120724. doi: 10.1038/nrc3310. PubMed PMID: 22825217.
19. Sawin K E, LeGuellec K, Philippe M, Mitchison T J. Mitotic spindle organization by a plus-end-directed microtubule motor. Nature. 1992; 359(6395):540-3. doi: 10.1038/359540a0. PubMed PMID: 1406972.
20. Rusan N M, Tulu U S, Fagerstrom C, Wadsworth P. Reorganization of the microtubule array in prophase/prometaphase requires cytoplasmic dynein-dependent microtubule transport. The Journal of cell biology. 2002; 158(6):997-1003. Epub 20020916. doi: 10.1083/jcb.200204109. PubMed PMID: 12235119; PMCID: PMC2173209.
21. Hoyt M A, He L, Loo K K, Saunders W S. Two Saccharomyces cerevisiae kinesin-related gene products required for mitotic spindle assembly. The Journal of cell biology. 1992; 118(1):109-20. doi: 10.1083/jcb.118.1.109. PubMed PMID: 1618897; PMCID: PMC2289527.
22. Kapoor T M, Mayer T U, Coughlin M L, Mitchison T J. Probing spindle assembly mechanisms with monastrol, a small molecule inhibitor of the mitotic kinesin, Eg5. The Journal of cell biology. 2000; 150(5):975-88. Epub 2000/09/07. PubMed PMID: 10973989; PMCID: PubMed PMID: 10973989; PubMed Central PMC2175262.
23. Heck M M, Pereira A, Pesavento P, Yannoni Y, Spradling A C, Goldstein L S. The kinesin-like protein KLP61F is essential for mitosis in Drosophila. The Journal of cell biology. 1993; 123(3):665-79. doi: 10.1083/jcb.123.3.665. PubMed PMID: 8227131; PMCID: PMC2200134.
24. Blangy A, Lane H A, d'Hérin P, Harper M, Kress M, Nigg E A. Phosphorylation by p34cdc2 regulates spindle association of human Eg5, a kinesin-related motor essential for bipolar spindle formation in vivo. Cell. 1995; 83(7):1159-69. doi: 10.1016/0092-8674(95)90142-6. PubMed PMID: 8548803.
25. Bannigan A, Scheible W R, Lukowitz W, Fagerstrom C, Wadsworth P, Somerville C, Baskin T I. A conserved role for kinesin-5 in plant mitosis. J Cell Sci. 2007; 120(Pt 16):2819-27. Epub 20070724. doi: 10.1242/jcs.009506. PubMed PMID: 17652157.
26. Mayer T U, Kapoor T M, Haggarty S J, King R W, Schreiber S L, Mitchison T J. Small molecule inhibitor of mitotic spindle bipolarity identified in a phenotype-based screen. Science. 1999; 286(5441):971-4. doi: 10.1126/science.286.5441.971. PubMed PMID: 10542155.
27. Gartner M, Sunder-Plassmann N, Seiler J, Utz M, Vernos I, Surrey T, Giannis A. Development and biological evaluation of potent and specific inhibitors of mitotic Kinesin Eg5. Chembiochem: a European journal of chemical biology. 2005; 6(7):1173-7. doi: 10.1002/cbic.200500005. PubMed PMID: 15912555.
28. Skoufias D A, DeBonis S, Saoudi Y, Lebeau L, Crevel I, Cross R, Wade R H, Hackney D, Kozielski F. S-trityl-L-cysteine is a reversible, tight binding inhibitor of the human kinesin Eg5 that specifically blocks mitotic progression. The Journal of biological chemistry. 2006; 281(26):17559-69. Epub 2006/03/02. doi: 10.1074/jbc.M511735200. PubMed PMID: 16507573; PMCID: PubMed PMID: 16507573.
29. Huszar D, Theoclitou M E, Skolnik J, Herbst R. Kinesin motor proteins as targets for cancer therapy. Cancer Metastasis Rev. 2009; 28(1-2):197-208. doi: 10.1007/s10555-009-9185-8. PubMed PMID: 19156502.
30. Filanesib (ARRY-520) 2015 [cited 2015 March 29]. Available from: arraybiopharma.com/product-pipeline/filanesib. 31. Liu X, Gong H, Huang K. Oncogenic role of kinesin proteins and targeting kinesin therapy. Cancer Sci. 2013; 104(6):651-6. Epub 20130404. doi: 10.1111/cas.12138. PubMed PMID: 23438337; PMCID: PMC7657121.
32. Luo L, Parrish C A, Nevins N, McNulty D E, Chaudhari A M, Carson J D, Sudakin V, Shaw A N, Lehr R, Zhao H, Sweitzer S, Lad L, Wood K W, Sakowicz R, Annan R S, Huang P S, Jackson J R, Dhanak D, Copeland R A, Auger K R. ATP-competitive inhibitors of the mitotic kinesin KSP that function via an allosteric mechanism. Nature chemical biology. 2007; 3(11):722-6. doi: 10.1038/nchembio.2007.34.
33. Holen K D, Belani C P, Wilding G, Ramalingam S, Volkman J L, Ramanathan R K, Vasist L S, Bowen C J, Hodge J P, Dar M M, Ho PTC. A first in human study of SB-743921, a kinesin spindle protein inhibitor, to determine pharmacokinetics, biologic effects and establish a recommended phase II dose. Cancer Chemotherapy and Pharmacology. 2011; 67(2):447-54. doi: 10.1007/s00280-010-1346-5.
34. Lee C W, Bélanger K, Rao S C, Petrella T M, Tozer R G, Wood L, Savage K J, Eisenhauer E A, Synold T W, Wainman N, Seymour L. A phase II study of ispinesib (SB-715992) in patients with metastatic or recurrent malignant melanoma: a National Cancer Institute of Canada Clinical Trials Group trial. Invest New Drugs. 2008; 26(3):249-55. Epub 20071026. doi: 10.1007/s10637-007-9097-9. PubMed PMID: 17962907.
35. Souid A K, Dubowy R L, Ingle A M, Conlan M G, Sun J, Blaney S M, Adamson P C. A pediatric phase I trial and pharmacokinetic study of ispinesib: a Children's Oncology Group phase I consortium study. Pediatr Blood Cancer. 2010; 55(7):1323-8. Epub 2010/08/17. doi: 10.1002/pbc.22609. PubMed PMID: 20712019; PMCID: PMC3053384.
36. Blagden S P, Molife L R, Seebaran A, Payne M, Reid A H, Protheroe A S, Vasist L S, Williams D D, Bowen C, Kathman S J, Hodge J P, Dar M M, de Bono J S, Middleton M R. A phase I trial of ispinesib, a kinesin spindle protein inhibitor, with docetaxel in patients with advanced solid tumours. Br J Cancer. 2008; 98(5):894-9. Epub 2008/03/06. doi: 10.1038/sj.bjc.6604264. PubMed PMID: 18319713; PMCID: PubMed PMID: 18319713; PubMed Central PMCID: PMC2266864.
37. Tang P A, Siu L L, Chen E X, Hotte S J, Chia S, Schwarz J K, Pond G R, Johnson C, Colevas A D, Synold T W, Vasist L S, Winquist E. Phase II study of ispinesib in recurrent or metastatic squamous cell carcinoma of the head and neck. Invest New Drugs. 2008; 26(3):257-64. Epub 20071124. doi: 10.1007/s10637-007-9098-8. PubMed PMID: 18038218.
38. Gomez H L, Philco M, Pimentel P, Kiyan M, Monsalvo M L, Conlan M G, Saikali K G, Chen M M, Seroogy J J, Wolff A A, Escandon R D. Phase I dose-escalation and pharmacokinetic study of ispinesib, a kinesin spindle protein inhibitor, administered on days 1 and 15 of a 28-day schedule in patients with no prior treatment for advanced breast cancer. Anti-cancer drugs. 2012; 23(3):335-41. Epub 2011/11/30. doi: 10.1097/CAD.0b013e32834e74d6. PubMed PMID: 22123335; PMCID: PubMed PMID: 22123335.
39. Beer T M, Goldman B, Synold T W, Ryan C W, Vasist L S, Van Veldhuizen P J, Jr., Dakhil S R, Lara P N, Jr., Drelichman A, Hussain M H, Crawford E D. Southwest Oncology Group phase II study of ispinesib in androgen-independent prostate cancer previously treated with taxanes. Clin Genitourin Cancer. 2008; 6(2):103-9. doi: 10.3816/CGC.2008.n.016. PubMed PMID: 18824433.
40. Lee R T, Beekman K E, Hussain M, Davis N B, Clark J I, Thomas S P, Nichols K F, Stadler W M. A University of Chicago consortium phase II trial of SB-715992 in advanced renal cell cancer. Clin Genitourin Cancer. 2008; 6(1):21-4. doi: 10.3816/CGC.2008.n.003. PubMed PMID: 18501078; PMCID: PMC2704464.
41. Burris H A, 3rd, Jones S F, Williams D D, Kathman S J, Hodge J P, Pandite L, Ho P T, Boerner S A, Lorusso P. A phase I study of ispinesib, a kinesin spindle protein inhibitor, administered weekly for three consecutive weeks of a 28-day cycle in patients with solid tumors. Invest New Drugs. 2011; 29(3):467-72. Epub 2010/01/14. doi: 10.1007/s10637-009-9374-x. PubMed PMID: 20069338; PMCID: PubMed PMID: 20069338.
42. O'Connor O A, Gerecitano J, Van Deventer H, Hainsworth J, Zullo K M, Saikali K, Seroogy J, Wolff A, Escandón R. The addition of granulocyte-colony stimulating factor shifts the dose limiting toxicity and markedly increases the maximum tolerated dose and activity of the kinesin spindle protein inhibitor SB-743921 in patients with relapsed or refractory lymphoma: results of an international, multicenter phase I/II study. Leuk Lymphoma. 2015; 56(9):2585-91. Epub 20150911. doi: 10.3109/10428194.2015.1004167. PubMed PMID: 25665464.
43. Knox J J, Gill S, Synold T W, Biagi J J, Major P, Feld R, Cripps C, Wainman N, Eisenhauer E, Seymour L. A phase II and pharmacokinetic study of SB-715992, in patients with metastatic hepatocellular carcinoma: a study of the National Cancer Institute of Canada Clinical Trials Group (NCIC CTG IND.168). Invest New Drugs. 2008; 26(3):265-72. Epub 20080115. doi: 10.1007/s10637-007-9103-2. PubMed PMID: 18196204.
44. Shah J J, Kaufman J L, Zonder J A, Cohen A D, Bensinger W I, Hilder B W, Rush S A, Walker D H, Tunquist B J, Litwiler K S, Ptaszynski M, Orlowski R Z, Lonial S. A Phase 1 and 2 study of Filanesib alone and in combination with low-dose dexamethasone in relapsed/refractory multiple myeloma. Cancer. 2017; 123(23):4617-30. Epub 20170817. doi: 10.1002/cncr.30892. PubMed PMID: 28817190; PMCID: PMC5856158.
45. Lee H C, Shah J J, Feng L, Manasanch E E, Lu R, Morphey A, Crumpton B, Patel K K, Wang M L, Alexanian R, Thomas S K, Weber D M, Orlowski R Z. A phase 1 study of filanesib, carfilzomib, and dexamethasone in patients with relapsed and/or refractory multiple myeloma. Blood Cancer J. 2019; 9(10):80. Epub 20191001. doi: 10.1038/s41408-019-0240-6. PubMed PMID: 31575851; PMCID: PMC6773683.
46. Khoury H J, Garcia-Manero G, Borthakur G, Kadia T, Foudray M C, Arellano M, Langston A, Bethelmie-Bryan B, Rush S, Litwiler K, Karan S, Simmons H, Marcus A I, Ptaszynski M, Kantarjian H. A phase 1 dose-escalation study of ARRY-520, a kinesin spindle protein inhibitor, in patients with advanced myeloid leukemias. Cancer. 2012; 118(14):3556-64. Epub 20111202. doi: 10.1002/cncr.26664. PubMed PMID: 22139909; PMCID: PMC4984525.
47. Chari A, Htut M, Zonder J A, Fay J W, Jakubowiak A J, Levy J B, Lau K, Burt S M, Tunquist B J, Hilder B W, Rush S A, Walker D H, Ptaszynski M, Kaufman J L. A phase 1 dose-escalation study of filanesib plus bortezomib and dexamethasone in patients with recurrent/refractory multiple myeloma. Cancer. 2016; 122(21):3327-35. Epub 20160719. doi: 10.1002/cncr.30174. PubMed PMID: 27433944; PMCID: PMC6857452.
48. LoRusso P M, Goncalves P H, Casetta L, Carter J A, Litwiler K, Roseberry D, Rush S, Schreiber J, Simmons H M, Ptaszynski M, Sausville E A. First-in-human phase 1 study of filanesib (ARRY-520), a kinesin spindle protein inhibitor, in patients with advanced solid tumors. Investigational new drugs. 2015; 33(2):440-9. Epub 20150217. doi: 10.1007/s10637-015-0211-0. PubMed PMID: 25684345.
49. Drechsler H, McAinsh A D. Kinesin-12 motors cooperate to suppress microtubule catastrophes and drive the formation of parallel microtubule bundles. Proc Natl Acad Sci USA. 2016; 113(12):E1635-44. Epub 20160311. doi: 10.1073/pnas.1516370113. PubMed PMID: 26969727; PMCID: PMC4812750.
50. Mann B J, Balchand S K, Wadsworth P. Regulation of Kif15 localization and motility by the C-terminus of TPX2 and microtubule dynamics. Molecular Biology of the Cell. 2017; 28(1):65-75. Epub 20161116. doi: 10.1091/mbc.E16-06-0476. PubMed PMID: 27852894; PMCID: PMC5221630.
51. Sturgill E G, Das D K, Takizawa Y, Shin Y, Collier S E, Ohi M D, Hwang W, Lang M J, Ohi R. Kinesin-12 Kif15 targets kinetochore fibers through an intrinsic two-step mechanism. Curr Biol. 2014; 24(19):2307-13. Epub 20140925. doi: 10.1016/j.cub.2014.08.022. PubMed PMID: 25264249; PMCID: PMC4207087.
52. Drechsler H, McHugh T, Singleton M R, Carter N J, McAinsh A D. The Kinesin-12 Kif15 is a processive track-switching tetramer. Elife. 2014; 3:e01724. Epub 20140325. doi: 10.7554/eLife.01724. PubMed PMID: 24668168; PMCID: PMC3965211.
53. Sturgill E G, Ohi R. Kinesin-12 differentially affects spindle assembly depending on its microtubule substrate. Curr Biol. 2013; 23(14):1280-90. Epub 20130620. doi: 10.1016/j.cub.2013.05.043. PubMed PMID: 23791727; PMCID: PMC3725213.
54. Liang B, Zheng W, Fang L, Wu L, Zhou F, Yin X, Yu X, Zou Z. Overexpressed targeting protein for Xklp2 (TPX2) serves as a promising prognostic marker and therapeutic target for gastric cancer. Cancer biology & therapy. 2016; 17(8):824-32. Epub 20160617. doi: 10.1080/15384047.2016.1195046. PubMed PMID: 27314162; PMCID: PMC5004702.
55. Sanchez-Pulido L, Perez L, Kuhn S, Vernos I, Andrade-Navarro M A. The C-terminal domain of TPX2 is made of alpha-helical tandem repeats. BMC Struct Biol. 2016; 16(1):17. Epub 20161026. doi: 10.1186/s12900-016-0070-8. PubMed PMID: 27782824; PMCID: PMC5080731.
56. Miwa T, Kokuryo T, Yokoyama Y, Yamaguchi J, Nagino M. Therapeutic potential of targeting protein for Xklp2 silencing for pancreatic cancer. Cancer Med. 2015; 4(7):1091-100. Epub 20150427. doi: 10.1002/cam4.453. PubMed PMID: 25914189; PMCID: PMC4529347.
57. Wittmann T, Boleti H, Antony C, Karsenti E, Vernos I. Localization of the kinesin-like protein Xklp2 to spindle poles requires a leucine zipper, a microtubule-associated protein, and dynein. The Journal of cell biology. 1998; 143(3):673-85. doi: 10.1083/jcb.143.3.673. PubMed PMID: 9813089; PMCID: PMC2148133.
58. Wittmann T, Wilm M, Karsenti E, Vernos I. TPX2, A novel xenopus MAP involved in spindle pole organization. The Journal of cell biology. 2000; 149(7):1405-18. doi: 10.1083/jcb.149.7.1405. PubMed PMID: 10871281; PMCID: PMC2175143.
59. van Heesbeen R, Raaijmakers J A, Tanenbaum M E, Halim V A, Lelieveld D, Lieftink C, Heck AJR, Egan D A, Medema R H. Aurora A, MCAK, and Kifl8b promote Eg5-independent spindle formation. Chromosoma. 2017; 126(4):473-86. Epub 20160629. doi: 10.1007/s00412-016-0607-4. PubMed PMID: 27354041; PMCID: PMC5509784.
60. Raaijmakers J A, van Heesbeen R G, Meaders J L, Geers E F, Fernandez-Garcia B, Medema R H, Tanenbaum M E. Nuclear envelope-associated dynein drives prophase centrosome separation and enables Eg5-independent bipolar spindle formation. Embo j. 2012; 31(21):4179-90. Epub 20121002. doi: 10.1038/emboj.2012.272. PubMed PMID: 23034402; PMCID: PMC3492733.
61. van Heesbeen Roy GHP, Tanenbaum Marvin E, Medema René H. Balanced Activity of Three Mitotic Motors Is Required for Bipolar Spindle Assembly and Chromosome Segregation. Cell Reports. 2014; 8(4):948-56. doi: doi.org/10.1016/j.celrep.2014.07.015.
62. Lucanus A J, Yip G W. Kinesin superfamily: roles in breast cancer, patient prognosis and therapeutics. Oncogene. 2018; 37(7):833-8. Epub 20171023. doi: 10.1038/onc.2017.406. PubMed PMID: 29059174.
63. Marquis C, Fonseca C L, Queen K A, Wood L, Vandal S E, Malaby HLH, Clayton J E, Stumpff J. Chromosomally unstable tumor cells specifically require KIF18A for proliferation. Nature communications. 2021; 12(1):1213. Epub 20210222. doi: 10.1038/s41467-021-21447-2. PubMed PMID: 33619254; PMCID: PMC7900194.
64. Kitzen J J, de Jonge M J, Verweij J. Aurora kinase inhibitors. Crit Rev Oncol Hematol. 2010; 73(2):99-110. Epub 20090414. doi: 10.1016/j.critrevonc.2009.03.009. PubMed PMID: 19369091.
65. Yan M, Wang C, He B, Yang M, Tong M, Long Z, Liu B, Peng F, Xu L, Zhang Y, Liang D, Lei H, Subrata S, Kelley K W, Lam E W, Jin B, Liu Q. Aurora-A Kinase: A Potent Oncogene and Target for Cancer Therapy. Med Res Rev. 2016; 36(6):1036-79. Epub 20160713. doi: 10.1002/med.21399. PubMed PMID: 27406026.
66. Du R, Huang C, Liu K, Li X, Dong Z. Targeting AURKA in Cancer: molecular mechanisms and opportunities for Cancer therapy. Mol Cancer. 2021; 20(1):15. Epub 2021/01/17. doi: 10.1186/s12943-020-01305-3. PubMed PMID: 33451333; PMCID: PMC7809767.
67. Falchook G S, Bastida C C, Kurzrock R. Aurora Kinase Inhibitors in Oncology Clinical Trials: Current State of the Progress. Semin Oncol. 2015; 42(6):832-48. Epub 2015/11/29. doi: 10.1053/j.seminoncol.2015.09.022. PubMed PMID: 26615129.
68. Tang A, Gao K, Chu L, Zhang R, Yang J, Zheng J. Aurora kinases: novel therapy targets in cancers. Oncotarget. 2017; 8(14):23937-54. Epub 2017/02/02. doi: 10.18632/oncotarget.14893. PubMed PMID: 28147341; PMCID: PMC5410356.
69. Replogle J M, Zhou W, Amaro A E, McFarland J M, Villalobos-Ortiz M, Ryan J, Letai A, Yilmaz O, Sheltzer J, Lippard S J, Ben-David U, Amon A. Aneuploidy increases resistance to chemotherapeutics by antagonizing cell division. Proc Natl Acad Sci USA. 2020; 117(48):30566-76. Epub 20201117. doi: 10.1073/pnas.2009506117. PubMed PMID: 33203674; PMCID: PMC7720170.
70. Cohen-Sharir Y, McFarland J M, Abdusamad M, Marquis C, Bernhard S V, Kazachkova M, Tang H, Ippolito M R, Lane K, Zerbib J, Malaby HLH, Jones A, Stautmeister L M, Bockaj I, Wardenaar R, Lyons N, Nagaraja A, Bass A J, Spierings DCJ, Foijer F, Beroukhim R, Santaguida S, Golub T R, Stumpff J, Storchovi Z, Ben-David U. Aneuploidy renders cancer cells vulnerable to mitotic checkpoint inhibition. Nature. 2021; 590(7846):486-91. Epub 20210127. doi: 10.1038/s41586-020-03114-6. PubMed PMID: 33505028; PMCID: PMC8262644.
71. Das P M, Bast R C, Jr. Early detection of ovarian cancer. Biomarkers in medicine. 2008; 2(3):291-303. doi: 10.2217/17520363.2.3.291. PubMed PMID: 20477415; PMCID: 2930760.
72. Lowe K A, Chia V M, Taylor A, O'Malley C, Kelsh M, Mohamed M, Mowat F S, Goff B. An international assessment of ovarian cancer incidence and mortality. Gynecologic oncology. 2013; 130(1):107-14. Epub 2013/04/06. doi: 10.1016/j.ygyno.2013.03.026. PubMed PMID: 23558050.
73. Meier J C, Haendler B, Seidel H, Groth P, Adams R, Ziegelbauer K, Kreft B, Beckmann G, Sommer A, Kopitz C. Knockdown of platinum-induced growth differentiation factor 15 abrogates p27-mediated tumor growth delay in the chemoresistant ovarian cancer model A2780cis. Cancer Med. 2015; 4(2):253-67. Epub 2014/12/11. doi: 10.1002/cam4.354. PubMed PMID: 25490861; PMCID: Pmc4329009.
74. Rein B J, Gupta S, Dada R, Safi J, Michener C, Agarwal A. Potential markers for detection and monitoring of ovarian cancer. J Oncol. 2011; 2011:475983. doi: 10.1155/2011/475983. PubMed PMID: 21577260; PMCID: 3090619.
75. Coleman R L, Monk B J, Sood A K, Herzog T J. Latest research and treatment of advanced-stage epithelial ovarian cancer. Nature reviews Clinical oncology. 2013; 10(4):211-24. Epub 2013/02/06. doi: 10.1038/nrclinonc.2013.5. PubMed PMID: 23381004; PMCID: 3786558.
76. Coward J I, Middleton K, Murphy F. New perspectives on targeted therapy in ovarian cancer. Int J Womens Health. 2015; 7:189-203. Epub 2015/02/14. doi: 10.2147/IJWH.S52379. PubMed PMID: 25678824; PMCID: 4324539.
77. Jelovac D, Armstrong D K. Recent progress in the diagnosis and treatment of ovarian cancer. C A Cancer J Clin. 2011; 61(3):183-203. Epub 2011/04/28. doi: 10.3322/caac.20113. PubMed PMTD: 21521830; PMCTD: 3576854.
78. Drescher C W, Hawley S, Thorpe J D, Marticke S, McIntosh M, Gambhir S S, Urban N. Impact of screening test performance and cost on mortality reduction and cost-effectiveness of multimodal ovarian cancer screening. Cancer prevention research. 2012; 5(8):1015-24. doi: 10.1158/1940-6207.CAPR-11-0468. PubMed PMID: 22750949; PMCID: 3729263.
79. van Nagell J R, Jr., Gallion H H, Pavlik E J, DePriest P D. Ovarian cancer screening. Cancer. 1995; 76(10 Suppl):2086-91. PubMed PMID: 8635005.
80. Weroha S J, Becker M A, Enderica-Gonzalez S, Harrington S C, Oberg A L, Maurer M J, Perkins S E, AlHilli M, Butler K A, McKinstry S, Fink S, Jenkins R B, Hou X, Kalli K R, Goodman K M, Sarkaria J N, Karlan B Y, Kumar A, Kaufmann S H, Hartmann L C, Haluska P. Tumorgrafts as in vivo surrogates for women with ovarian cancer. Clin Cancer Res. 2014; 20(5):1288-97. doi: 10.1158/1078-0432.CCR-13-2611. PubMed PMID: 24398046; PMCID: PubMed PMID: 24398046; PubMed Central PMCID: PMC3947430.
81. Siegel R L, Miller K D, Jemal A. Cancer statistics, 2015. C A Cancer J Clin. 2015; 65(1):5-29. doi: 10.3322/caac.21254. PubMed PMID: 25559415.
82. Arndt C A, Rose P S, Folpe A L, Laack N N. Common musculoskeletal tumors of childhood and adolescence. Mayo Clin Proc. 2012; 87(5):475-87. doi: 10.1016/j.mayocp.2012.01.015. PubMed PMID: 22560526; PMCID: PMC3538469.
83. Balamuth N J, Womer R B. Ewing's sarcoma. Lancet Oncol. 2010; 11(2):184-92. Epub 2010/02/16. doi: 10.1016/s1470-2045(09)70286-4. PubMed PMID: 20152770.
84. Esiashvili N, Goodman M, Marcus R B, Jr. Changes in incidence and survival of Ewing sarcoma patients over the past 3 decades: Surveillance Epidemiology and End Results data. J Pediatr Hematol Oncol. 2008; 30(6):425-30. Epub 2008/06/06. doi: 10.1097/MPH.0b013e31816e22f3. PubMed PMID: 18525458.
85. Riggi N, Suva M L, Stamenkovic I. Ewing's Sarcoma. N Engl J Med. 2021; 384(2):154-64. doi: 10.1056/NEJMra2028910. PubMed PMID: 33497548.
86. Reinemann D N, Sturgill E G, Das D K, Degen M S, VWr6s Z, Hwang W, Ohi R, Lang M J. Collective Force Regulation in Anti-parallel Microtubule Gliding by Dimeric Kif15 Kinesin Motors. Current Biology. 2017; 27(18):2810-20.e6. doi: 10.1016/j.cub.2017.08.018.
87. Tanenbaum M E, Macurek L, Janssen A, Geers E F, Alvarez-Fernandez M, Medema R H. Kif15 cooperates with eg5 to promote bipolar spindle assembly. Curr Biol. 2009; 19(20):1703-11. Epub 2009/10/13. doi: 10.1016/j.cub.2009.08.027. PubMed PMID: 19818618; PMCID: PubMed PMID: 19818618.
88. Toso A, Winter J R, Garrod A J, Amaro A C, Meraldi P, McAinsh A D. Kinetochore-generated pushing forces separate centrosomes during bipolar spindle assembly. The Journal of cell biology. 2009; 184(3):365-72. doi: 10.1083/jcb.200809055. PubMed PMID: 19204145; PMCID: PMC2646558.
89. Gibbs B K, Wates R J, Roy A, Schoenen F J, Douglas J, McDonald P, Whitaker A, Ndi C, Pathak H, Brinker A, Harned L, Neuenswander S, Broward M A, Freudenthal B, Godwin A K. The Identification of Chemotypes for the Inhibition of KIF15-TPX2 Protein-Protein Interaction for the Treatment of Epithelial Ovarian Cancer. In Preparation.
90. Fischer G, Rossmann M, Hyvonen M. Alternative modulation of protein-protein interactions by small molecules. Curr Opin Biotechnol. 2015; 35:78-85. Epub 20150515. doi: 10.1016/j.copbio.2015.04.006. PubMed PMID: 25935873; PMCID: PMC4728186.
91. Fang C J, Gui L, Zhang X, Moen D R, Li K, Frankowski K J, Lin H J, Schoenen F J, Chou T F. Evaluating p97 inhibitor analogues for their domain selectivity and potency against the p97-p47 complex. ChemMedChem. 2015; 10(1):52-6. Epub 20141106. doi: 10.1002/cmdc.201402420. PubMed PMID: 25377500; PMCID: PMC4280364.
92. Gui L, Zhang X, Li K, Frankowski K J, Li S, Wong D E, Moen D R, Porubsky P R, Lin H J, Schoenen F J, Chou T F. Evaluating p97 Inhibitor Analogues for Potency against p97-p37 and p97-Npl4-Ufdl Complexes. ChemMedChem. 2016; 11(9):953-7. Epub 20160404. doi: 10.1002/cmdc.201600036. PubMed PMID: 27043824; PMCID: PMC9049307.
93. Sethi G, Kwon Y, Burkhalter R J, Pathak H B, Madan R, McHugh S, Atay S, Murthy S, Tawfik O W, Godwin A K. PTN signaling: Components and mechanistic insights in human ovarian cancer. Molecular Carcinogenesis. 2015; 54(12):1772-85. Epub 20141121. doi: doi.org/10.1002/mc.22249. PubMed PMID: 25418856; PMCID: PMC4456343.
94. Shahin M S, Braly P, Rose P, Malpass T, Bailey H, Alvarez R D, Hodge J, Bowen C, Buller R. A phase II, open-label study of ispinesib (SB-715992) in patients with platinum/taxane refractory or resistant relapsed ovarian cancer. Journal of Clinical Oncology. 2007; 25(18_suppl):5562-. doi: 10.1200/jco.2007.25.18_suppl.5562.
95. Terribas E, Fernindez M, Mazuelas H, Fernindez-Rodriguez J, Biayna J, Blanco I, Bernal G, Ramos-Oliver I, Thomas C, Guha R, Zhang X, Gel B, Romagosa C, Ferrer M, Lizaro C, Serra E. KIF11 and KIF15 mitotic kinesins are potential therapeutic vulnerabilities for malignant peripheral nerve sheath tumors. Neurooncol Adv. 2020; 2(Suppl 1):i62-i74. Epub 20200104. doi: 10.1093/noajnl/vdz061. PubMed PMID: 32642733; PMCID: PMC7317059.
96. Milic B, Chakraborty A, Han K, Bassik M C, Block S M. KIF15 nanomechanics and kinesin inhibitors, with implications for cancer chemotherapeutics. Proc Natl Acad Sci USA. 2018; 115(20):E4613-e22. Epub 20180427. doi: 10.1073/pnas.1801242115. PubMed PMID: 29703754; PMCID: PMC5960320.
97. Sun R F, He N, Zhang G Y, Yu Z Y, Li L S, Ma Z J, Jiao Z Y. Combined inhibition of KIF11 and KIF15 as an effective therapeutic strategy for gastric cancer. Curr Cancer Drug Targets. 2022. Epub 20220616. doi: 10.2174/1568009622666220616122846. PubMed PMID: 35713129.
98. Dumas M E, Chen G Y, Kendrick N D, Xu G, Larsen S D, Jana S, Waterson A G, Bauer J A, Hancock W, Sulikowski G A, Ohi R. Dual inhibition of Kif15 by oxindole and quinazolinedione chemical probes. Bioorg Med Chem Lett. 2019; 29(2):148-54. Epub 20181204. doi: 10.1016/j.bmcl.2018.12.008. PubMed PMID: 30528696; PMCID: PMC6681659.
99. Schorpp K, Rothenaigner I, Salmina E, Reinshagen J, Low T, Brenke J K, Gopalakrishnan J, Tetko I V, Gul S, Hadian K. Identification of Small-Molecule Frequent Hitters from AlphaScreen High-Throughput Screens. Journal of biomolecular screening. 2014; 19(5):715-26. doi: 10.1177/1087057113516861. PubMed PMID: 24371213; PMCID: PubMed PMID: 24371213; PubMed Central PMCID: PMC4153540.
100. Duffy B C, Zhu L, Decornez H, Kitchen D B. Early phase drug discovery: cheminformatics and computational techniques in identifying lead series. Bioorganic & medicinal chemistry. 2012; 20(18):5324-42. Epub 20120505. doi: 10.1016/j.bmc.2012.04.062. PubMed PMID: 22938785.

TABLE 1

| | | | | | | NMR |
|---|---|---|---|---|---|---|
| | | | KIF15/TPX2 | His-GST | | Versus |
| | Compound | HTS | AlphaScreen; | AlphaScreen; | TruHits; | GST- |
| Compound Name | No. | Code | IC50 (μM) | IC50 (μM) | IC50 (μM) | KIF15 |
| 6-(4-ethylbenzyl)-2- | Compound | 747 | 32.2 ± 4.6 | 322 ± 6 | 393 ± 13 | No |
| (4-propylpiperazin- | 1 | | | | | |
| 1-yl)-5,6,7,8- | | | | | | |
| tetrahydropyrido | | | | | | |
| [4,3-d]pyrimidine | | | | | | |
| 6-(2,3-difluorobenzyl)- | Compound | 756 | 27.8 ± 4.7 | 386 ± 18 | 428 ± 38 | No |
| 2-(4-propylpiperazin- | 2 | | | | | |
| 1-yl)-5,6,7,8- | | | | | | |
| tetrahydropyrido | | | | | | |
| [4,3-d]pyrimidine | | | | | | |
| 6-(3,4-diethoxybenzyl)- | Compound | 737 | 42.2 ± 3.3 | 392 ± 14 | 510 ± 6 | No |
| 2-(4-propylpiperazin- | 3 | | | | | |
| 1-yl)-5,6,7,8- | | | | | | |
| tetrahydropyrido | | | | | | |
| [4,3-d]pyrimidine | | | | | | |
| 6-((5-bromothiophen- | Compound | 743 | 23 ± 1.6 | 286 ± 15 | 312 ± 79 | Hit |
| 2-yl)methyl)-2- | 4 | | | | | |
| (4-propylpiperazin- | | | | | | |
| 1-yl)-5,6,7,8- | | | | | | |
| tetrahydropyrido | | | | | | |
| [4,3-d]pyrimidine | | | | | | |
| 6-(3-bromobenzyl)-2- | Compound | 750 | 21 ± 0.05 | 173 ± 2 | 287 ± 8 | Hit |
| (4-propylpiperazin- | 5 | | | | | |
| 1-yl)-5,6,7,8- | | | | | | |
| tetrahydropyrido | | | | | | |
| [4,3-d]pyrimidine | | | | | | |
| 6-(4-bromobenzyl)-2- | Compound | 790 | 13.6 ± 1.2 | 598 ± 83 | 628 ± 35 | Hit |
| (4-propylpiperazin- | 6 | | | | | |
| 1-yl)-5,6,7,8- | | | | | | |
| tetrahydropyrido | | | | | | |
| [4,3-d]pyrimidine | | | | | | |
| 6-(3,4-diethoxybenzyl)- | Compound | 738 | 2.8 ± 0.3 | 149 ± 0.2 | 176 ± 9 | No |
| 2-(4-ethylpiperazin-1- | 7 | | | | | |
| yl)-5,6,7,8- | | | | | | |
| tetrahydropyrido | | | | | | |
| [4,3-d]pyrimidine | | | | | | |
| 2-(4-ethylpiperazin-1- | Compound | 762 | 2.4 ± 0.1 | 86 ± 2 | 120 ± 5.2 | No |
| yl)-6-(2,4,5- | 8 | | | | | |
| trimethoxybenzyl)- | | | | | | |
| 5,6,7,8- | | | | | | |
| tetrahydropyrido | | | | | | |
| [4,3-d]pyrimidine | | | | | | |
| 2-(4-ethylpiperazin-1- | Compound | 745 | 1.7 ± 0.1 | 141 ± 5 | 196 ± 0.1 | No |
| yl)-6-(2,3,4- | 9 | | | | | |
| trimethoxybenzyl)- | | | | | | |
| 5,6,7,8- | | | | | | |
| tetrahydropyrido | | | | | | |
| [4,3-d]pyrimidine | | | | | | |
| 6-(2-chlorobenzyl)-2- | Compound | 799 | 1.5 ± 0.01 | 92 ± 11 | 86 ± 16 | Hit |
| (4-ethylpiperazin-1- | 10 (A1) | | | | | |
| yl)-5,6,7,8- | | | | | | |
| tetrahydropyrido | | | | | | |
| [4,3-d]pyrimidine | | | | | | |
| 2-(4-ethylpiperazin-1- | Compound | 793 | 2.5 ± 0.8 | 329 ± 1.1 | 556 ± 38 | No |
| yl)-6-(3,4,5- | 11 | | | | | |
| trimethoxybenzyl)- | | | | | | |
| 5,6,7,8- | | | | | | |
| tetrahydropyrido | | | | | | |
| [4,3-d]pyrimidine | | | | | | |
| 6-(4-ethoxy-3- | Compound | 731 | 3.5 ± 0.5 | 218 ± 3 | 305 ± 13 | No |
| methoxybenzyl)- | 12 | | | | | |
| 2-(4-ethylpiperazin- | | | | | | |
| 1-yl)-5,6,7,8- | | | | | | |
| tetrahydropyrido | | | | | | |
| [4,3-d]pyrimidine | | | | | | |
| 6-(4-ethoxy-3- | Compound | 730 | 44.8 ± 0.2 | 490 ± 0.8 | 664 ± 103 | No |
| methoxybenzyl)- | 13 | | | | | |
| 2-(4-propylpiperazin- | | | | | | |
| 1-yl)-5,6,7,8- | | | | | | |
| tetrahydropyrido | | | | | | |
| [4,3-d]pyrimidine | | | | | | |
| 4-allyl-2-methyl- | Compound | 751 | 5.6 ± 0.3 | 68 ± 3 | 87 ± 2 | NA |
| N-(pyridin-2- | 14 (B1) | | | | | |
| ylmethyl)-4H- | | | | | | |
| thieno[3,2- | | | | | | |
| b]pyrrole-5- | | | | | | |
| carboxamide | | | | | | |
| 6-((4-bromothiophen- | Compound | NA | NA | NA | NA | Hit |
| 2-yl)methyl)-2- | 15 | | | | | |
| (4-propylpiperazin- | | | | | | |
| 1-yl)-5,6,7,8- | | | | | | |
| tetrahydropyrido | | | | | | |
| [4,3-d]pyrimidine | | | | | | |
| 6-(2,4-difluorobenzyl)- | Compound | NA | NA | NA | NA | No |
| 2-(4-ethylpiperazin- | 16 | | | | | |
| 1-yl)-5,6,7,8- | | | | | | |
| tetrahydropyrido | | | | | | |
| [4,3-d]pyrimidine | | | | | | |
| 6-(2,3-dimethoxybenzyl)- | Compound | NA | NA | NA | NA | No |
| 2-(4-ethylpiperazin-1- | 17 | | | | | |
| yl)-5,6,7,8- | | | | | | |
| tetrahydropyrido | | | | | | |
| [4,3-d]pyrimidine | | | | | | |
| 6-((4-bromothiophen- | Compound | NA | NA | NA | NA | Hit |
| 2-yl)methyl)-2- | 18 | | | | | |
| (4-ethylpiperazin-1- | | | | | | |
| yl)-5,6,7,8- | | | | | | |
| tetrahydropyrido | | | | | | |
| [4,3-d]pyrimidine | | | | | | |
| 2-(4-ethylpiperazin-1- | Compound | NA | NA | NA | NA | No |
| yl)-6-(4-isopropoxy-3- | 19 | | | | | |
| methoxybenzyl)-5,6,7,8- | | | | | | |
| tetrahydropyrido | | | | | | |
| [4,3-d]pyrimidine | | | | | | |
| 6-(3-ethoxybenzyl)-2- | Compound | NA | NA | NA | NA | No |
| (4-ethylpiperazin-1- | 20 | | | | | |
| yl)-5,6,7,8- | | | | | | |
| tetrahydropyrido | | | | | | |
| [4,3-d]pyrimidine | | | | | | |

1. A method of inhibiting KIF15, comprising:
administering a compound to the KIF15 in order to inhibit biological functionality of KIF15, wherein the compound includes a structure of Formula 1; 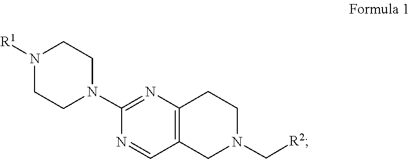
wherein:
R¹ and R² are each independently a non-hydrogen substituent, such that the compound inhibits biological functionality of KIF15,
or a stereoisomeric form or a mixture of stereoisomeric forms, prodrugs, or pharmaceutically acceptable salts of the compound.

2. The method of claim 1, wherein R¹ and R² each independently include a halogen, hydroxyl, alkoxy, straight aliphatic, branched aliphatic, cyclic aliphatic, substituted aliphatic, unsubstituted aliphatic, saturated aliphatic, unsaturated aliphatic, aromatic, polyaromatic, substituted aromatic, hetero-aromatic, hetero-polyaromatic, substituted hetero-polyaromatic, amine, primary amine, secondary amine, tertiary amine, aliphatic amine, carbonyl, carboxyl, amide, ester, phosphate, alkyl phosphate, phosphonate, alkyl phosphonate, carbamate, alkyl carbamate, amino alkyl carbamate, amino acid carbamate, amino acid, peptide, polypeptide, any aryl or cyclo with or without hetero atoms, each being substituted or unsubstituted, or combinations thereof.

3. The method of claim 1, wherein R¹ and R² each independently includes an alkyl, alkenyl, alkynyl, cycloalkyl, cycloalkenyl, cycloalkynyl, aryl, polyaryl, hetroaryl, polyhetroaryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof.

4. The method of claim 1, wherein R¹ includes a C1-C24 alkyl;
R² includes a phenyl, thiophenyl, thiazolyl, imidazolyl, furanyl, pyrrolyl, pyridinyl, pyridazinyl, quinolinyl, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, or morpholinyl, and quinuclidinyl, any substituted or unsubstituted with at least one R³; and
each R³ independently includes an alkyl, alkenyl, alkynyl, cycloalkyl, cycloalkenyl, cycloalkynyl, aryl, polyaryl, hetroaryl, polyhetroaryl, alkaryl, aralkyl, halo, hydroxyl, sulfhydryl, alkoxy, alkenyloxy, alkynyloxy, aryloxy, acyl, alkylcarbonyl, arylcarbonyl, acyloxy, alkoxycarbonyl, aryloxycarbonyl, halocarbonyl, alkylcarbonato, arylcarbonato, carboxy, carboxylato, carbamoyl, mono-(alkyl)-substituted carbamoyl, di-(alkyl)-substituted carbamoyl, mono-substituted arylcarbamoyl, thiocarbamoyl, carbamido, cyano, isocyano, cyanato, isocyanato, isothiocyanato, azido, formyl, thioformyl, amino, mono- and di-(alkyl)-substituted amino, mono- and di-(aryl)-substituted amino, alkylamido arylamido, imino, alkylimino, arylimino, nitro, nitroso, sulfo, sulfonato, alkylsulfanyl, arylsulfanyl, alkylsulfinyl, arylsulfinyl, alkylsulfonyl, arylsulfonyl, phosphono, phosphonato, phosphinato, phospho, phosphino, any aryl or cyclo with or without hetero atoms, each being substituted or unsubstituted, and combinations thereof.

5. The method of claim 1, wherein R¹ includes a C1-C6 alkyl;
R² includes phenyl, thiophenyl, furanyl, pyrrolyl, pyridinyl, or pyridazinyl, which is substituted with at least one R³; and
each R³ independently includes fluorine, chlorine, bromine, methyl, ethyl, propyl, isopropyl, butyl, hexyl, methoxy, ethoxy, propoxy, isopropoxy, butoxy, hexoxy, methyl ether, ethyl ether, propyl ether, isopropyl ether, butyl ether, or hexyl ether.

6. The method of claim 1, wherein R¹ is methyl, ethyl, or propyl;
R² includes phenyl or thiophenyl, which is substituted with at least one R³; and
each R³ independently includes fluorine, chlorine, bromine, methyl, ethyl, methoxy, ethoxy, propoxy, or isopropoxy.

7. The method of claim 6, wherein the R² includes phenyl and there are 1, 2, or 3 R³,
each R³ independently includes fluorine, chlorine, bromine, methyl, ethyl, methoxy, or ethoxy.

8. The method of claim 1, wherein the R¹ is methyl, ethyl, or propyl;
the R² includes thiophenyl; and
one R³ group including fluorine, chlorine, or bromine.

9. The method of claim 1, wherein the compound has a structure of one of the following: 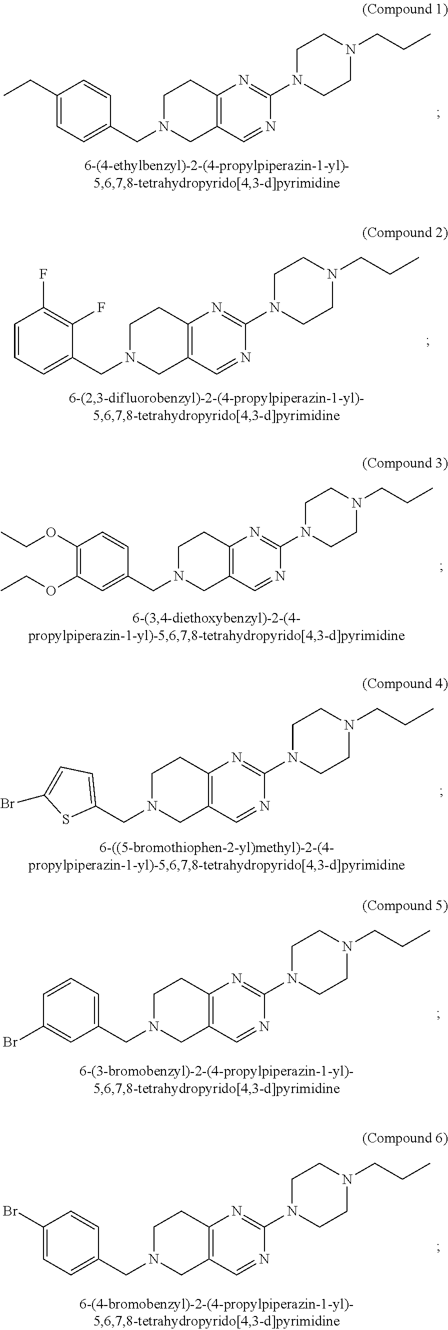 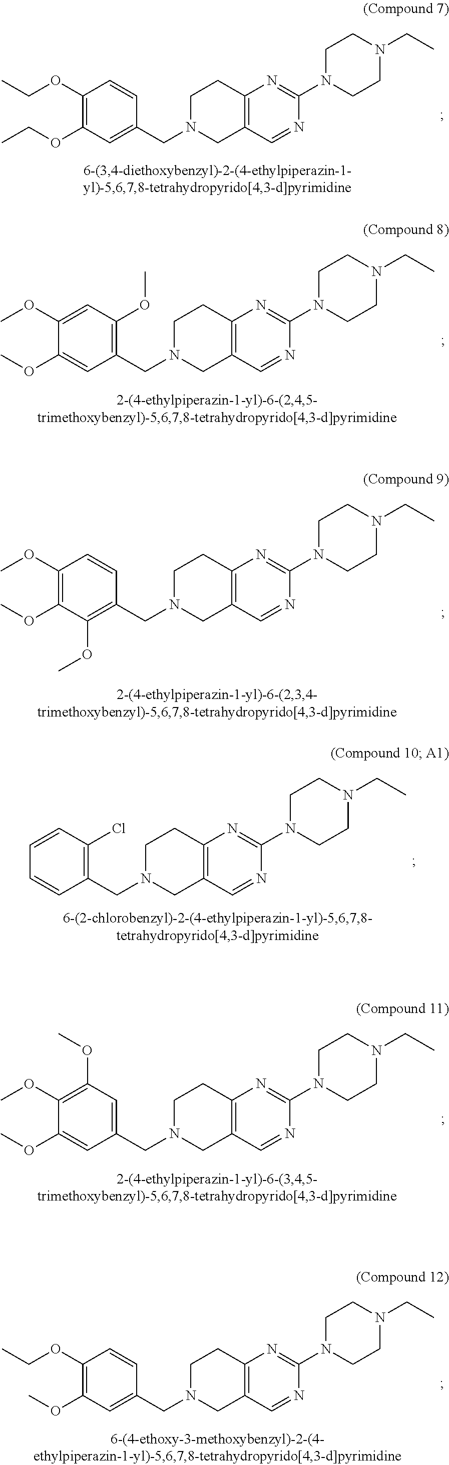 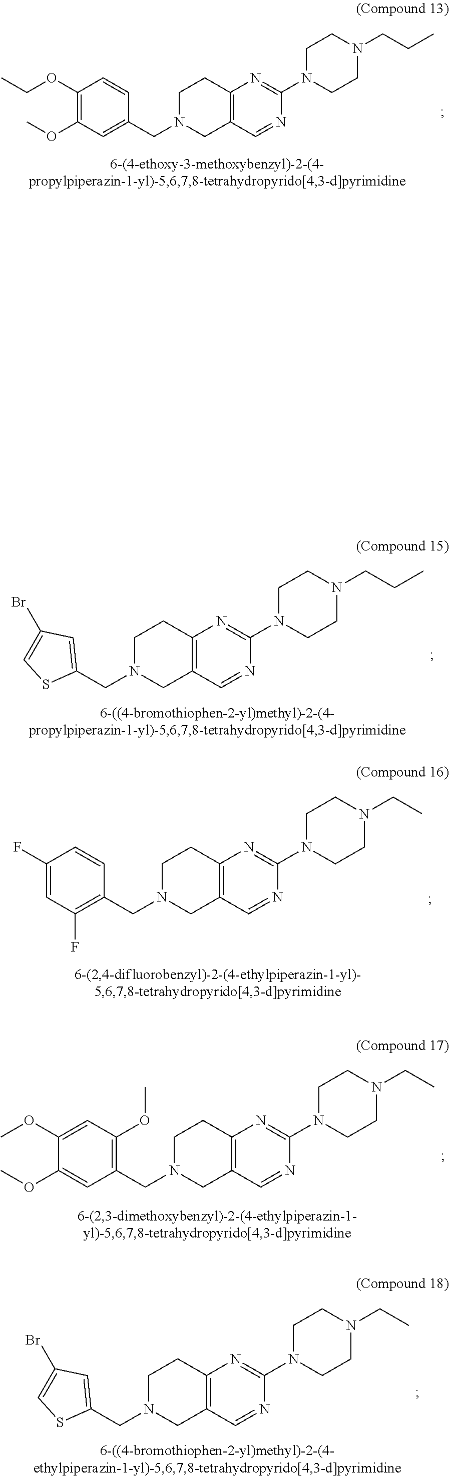 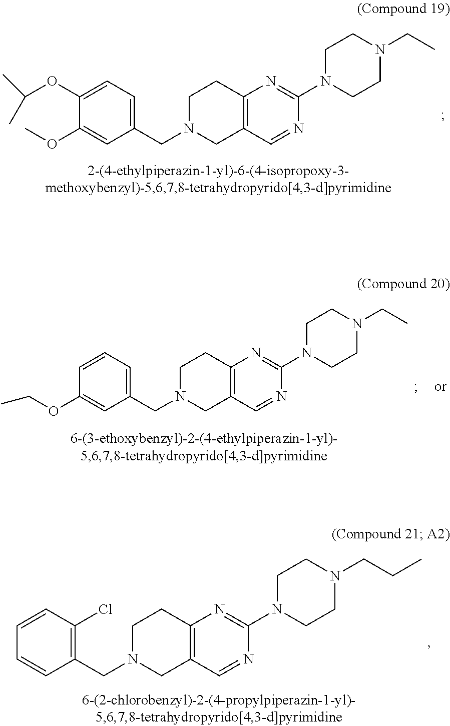
or a stereoisomeric form or a mixture of stereoisomeric forms, prodrugs, or pharmaceutically acceptable salts of the compound.

10. The method of claim 9, wherein the compound is one of Compounds 4, 5, 6, 10, 15, or 18.

11. The method of claim 1, wherein the compound is administered to the KIF15 in order to inhibit KIF15 from interacting with TPX2 so as to inhibit a protein-protein interaction therebetween.

12. The method of claim 1, wherein the administration of the compound to the KIF15 is in vitro.

13. The method of claim 1, wherein the administration of the compound to the KIF15 is in vivo.

14. The method of claim 1, wherein the administration of the compound to the KIF15 is by administering the compound to a subject having the KIF15, wherein the compound is administered in a therapeutically effective amount to treat a disease or disorder associated with KIF15 interacting with TPX2.

15. The method of claim 1, further comprising:
providing a subject having been diagnosed with a cancer or neoplasm; and
administering the compound to the subject in a therapeutically effective amount to treat the cancer or neoplasm.

16. The method of claim 15, wherein the cancer is selected from breast cancer, colorectal cancer, lung cancer, ovarian cancer, bone cancer, and prostate cancer.

17. The method of claim 15, wherein the cancer is a sarcoma selected from Ewing sarcoma (EWS), osteosarcoma, chondrosaroma, fibrosarcoma, synovial sarcoma, rhabdomysarcoma, angiosarcoma.

18. The method of claim 11, further comprising inhibiting KIF11 by administering a second compound that inhibits biological functionality of KIF11.

19. The method of claim 18, wherein the second compound includes an siRNA for KIF11, monastrol, Eg5 inhibitors, S-trityl-L-cysteine, ispinesib, filanesib, or a tail domain of KIF11 peptide.

20. A method of treating a cancer or neoplasm, comprising:
inhibiting biological functionality of KIF15 in the subject with a compound comprising a structure of Formula 1; 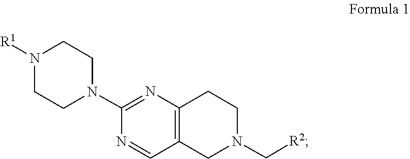
wherein:
R¹ and R² are each independently a non-hydrogen substituent, such that the compound inhibits biological functionality of KIF15,
or a stereoisomeric form or a mixture of stereoisomeric forms, prodrugs, or pharmaceutically acceptable salts of the compound.

21. The method of claim 20, comprising inhibiting biological functionality of KIF11 in a subject having the cancer or neoplasm.

22. The method of claim 21, comprising administering a combination of:
a KIF11 inhibitor selected from an siRNA for KIF11, monastrol, Eg5 inhibitors, S-trityl-L-cysteine, ispinesib, filanesib, or a tail domain of KIF11 peptide; and
the compound, wherein the compound includes:
R¹ includes a C1-C6 alkyl;
R² includes phenyl, thiophenyl, furanyl, pyrrolyl, pyridinyl, or pyridazinyl, which is substituted with at least one R³; and
each R³ is independently fluorine, chlorine, bromine, methyl, ethyl, propyl, isopropyl, butyl, hexyl, methoxy, ethoxy, propoxy, isopropoxy, butoxy, hexoxy, methyl ether, ethyl ether, propyl ether, isopropyl ether, butyl ether, or hexyl ether.

23. The method of claim 20, wherein R¹ is methyl, ethyl, or propyl;
R² includes phenyl or thiophenyl, which is substituted with at least one R³,
each R³ is independently fluorine, chlorine, bromine, methyl, ethyl, methoxy, ethoxy, propoxy, or isopropoxy.

24. The method of claim 23, wherein the R² includes phenyl and there are 1, 2, or 3 R³,
each R³ being fluorine, chlorine, bromine, methyl, ethyl, methoxy, or ethoxy.

25. The method of claim 20, wherein the R¹ is methyl, ethyl, or propyl;
the R² includes thiophenyl; and
one R³ group including fluorine, chlorine, or bromine.

26. The method of claim 20, wherein the compound has a structure of one of the following: 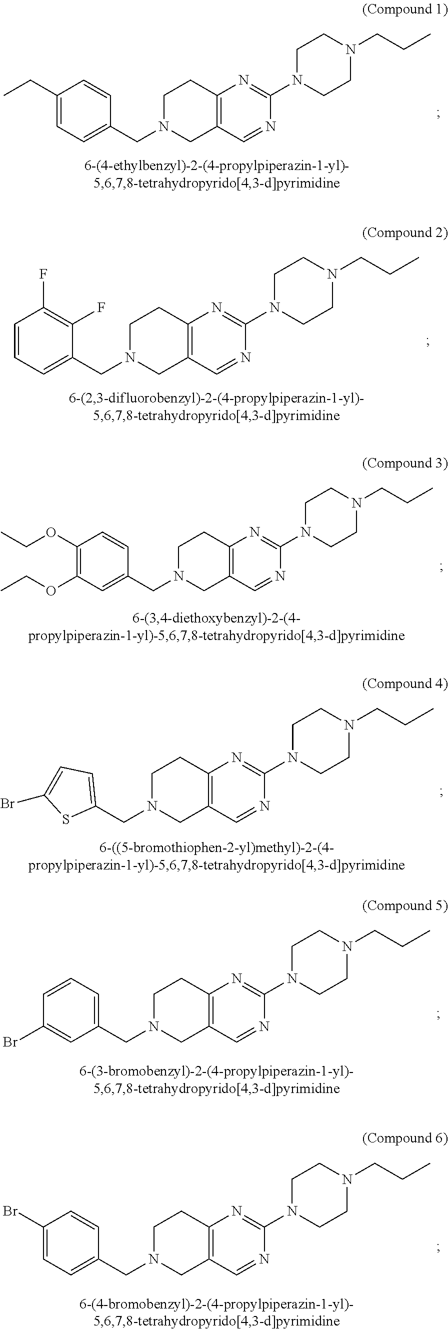 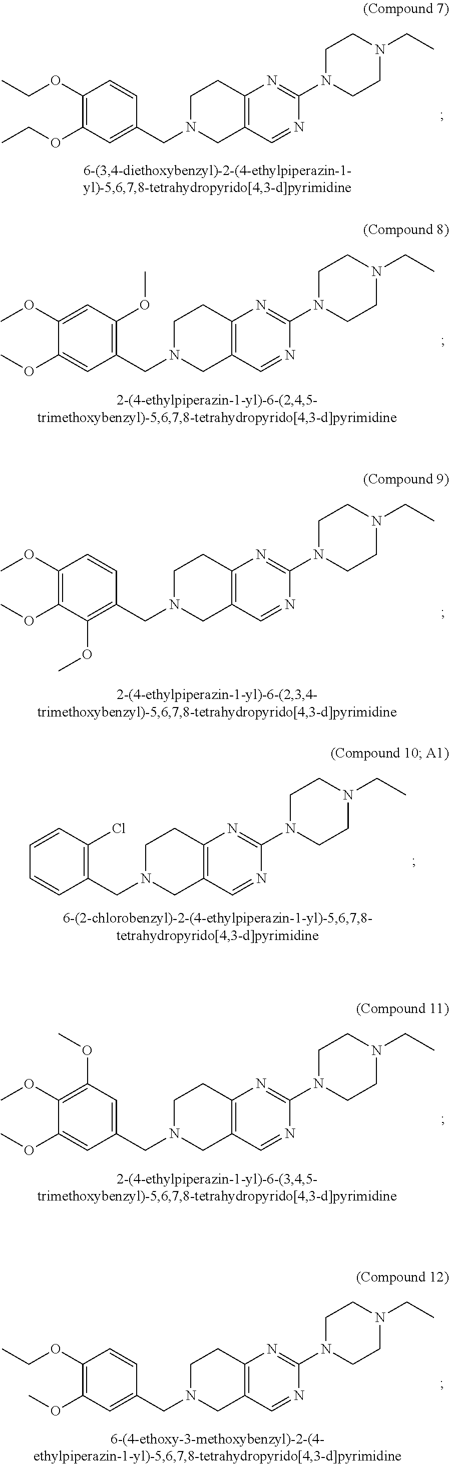 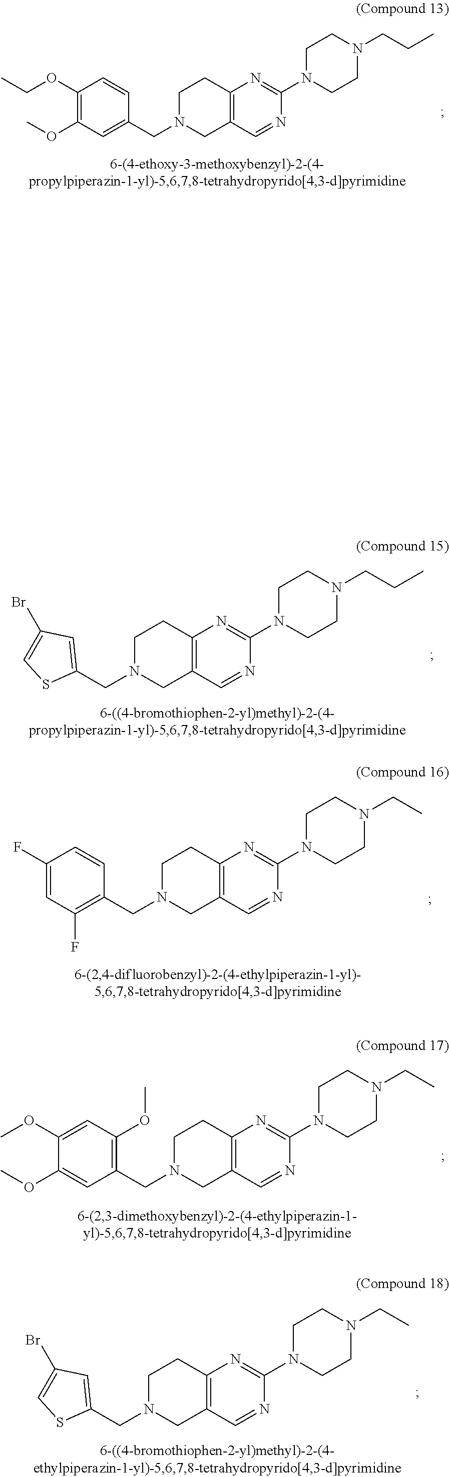 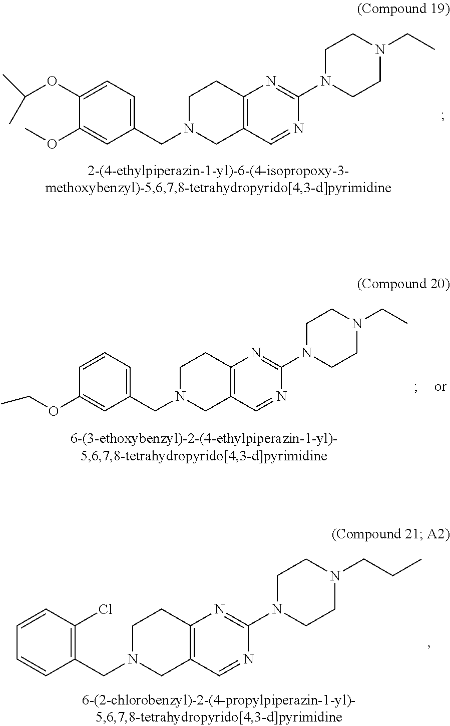
or a stereoisomeric form or a mixture of stereoisomeric forms, prodrugs, or pharmaceutically acceptable salts of the compound.

27. The method of claim 26, wherein the compound is one of Compounds 4, 5, 6, 10, 15, or 18.

28. A method of inhibiting KIF15, comprising:
administering a compound to the KIF15 in order to inhibit biological functionality of KIF15, wherein the compound includes a structure of: 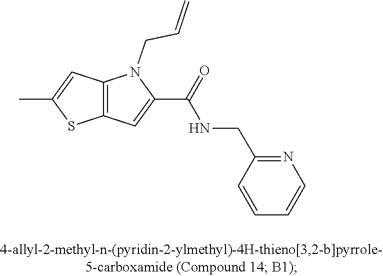 [SMILES: C=CCn1c(C(=O)NCc2ccccn2)cc2sc(C)cc21]
or a stereoisomeric form or a mixture of stereoisomeric forms, prodrugs, or pharmaceutically acceptable salts of the compound.

29. A method of treating a cancer or neoplasm, comprising:
inhibiting biological functionality of KIF11 in a subject having the cancer or neoplasm; and
inhibiting biological functionality of KIF15 in the subject with a compound comprising a structure of: 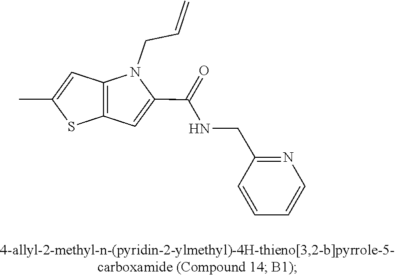 [SMILES: C=CCn1c(C(=O)NCc2ccccn2)cc2sc(C)cc21]
or a stereoisomeric form or a mixture of stereoisomeric forms, prodrugs, or pharmaceutically acceptable salts of the compound.
